SEQUENCE LISTING (80 sequences)

SEQ ID NO: 1 - DNA, 2160 bp, Artificial Sequence
  Promoter
ctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacc

SEQ ID NO: 2 - DNA, 774 bp, Artificial Sequence
  Terminator
cgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcga

SEQ ID NO: 3 - DNA, 15739 bp, Artificial Sequence
  Vector
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 4 - DNA, 11611 bp, Artificial Sequence
  Vector
agcttgcatgcctgcaggtcgagtggagatgtggagtgggcgcttacacagtacacgaggacttctagctagaaagaaggattacctctaaacaagtgtacctgtgcattctgggtaaacgactcataggagagttgtaaaaaagtttcggccggcgtattgggtgttacggagcattcactaggcaaccatgcatggttactattgtataccatcttagtaggaantgatttcgaggtttatacctacgatgaatgtgtgtcctgtaggcttgagagttcaaggaagaaacatgcaattatctttgcgaacccaggngctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagctctgctaagctattcttctcttcgccggagcctgnaaggcgttactaggttgcagtcaatgcattaatgcattgcagatgagctgtatctggaagaggtaaacccgaaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcgatcccgcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcatggtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggccagtggcttattactcagcccttctctctgcgtccgtccgtctctccgcatgccagaaagagtcaccggtcactgtacagagctcacgagttcgtcacatttttctacaaatggtggaggcggcggattttaggctcaagtcatgaccctctgggtcactccagaatcagctaggtcaacgaataaggatgattctataggaagatccaggcaccggtcaaccatgatctggacagatttgggagctcggtataagctctccacctatcttattctgtatagtttaggcttaaagtttatccaggagatgttgctgaagtcgatttgagtccacttcctcactggtagctatacgactttgatggtcgttgtaggggctgtattaggtctcgatcaaacacaaatagaattaaatggtactcgagtccactgaaggtggcttctccgtcttccgtagccgtgccgaaatccttacagcttgtgttgtgtgacttttggttacgccgtctgacttttgtggtgagctaactagagatcatgctatatctcctgatttaatacaatgctcatcataacattccacctggaactgctagcaacgtttgacttgcattgtgcaacgccctttgcagagctatcggatgatcaatagtgccacgttctaaattcaaccaacgcaggtgccccaagccttcgacatccggatgtatttcgaaaacctcatggcgattgcagtcctcagattcatgttcattccaatgctcattggtgaataaaaggttcacagggaataagttcaaactcgagatacttgagaatattgaaagccaaaggaccctctatgctccaagctagagtctcagcctggaaagcaaatccaaatgaagctatgctacctccaattcctcatcatcttatctataatacagagtcgaagaatatcctcttgacaccgctccgtcctccgacttcaataaggagcttactcctccttgacaccacccctccagttcttctcggcgttctggagggaggccttgtcggtcttgggctggccctggctgagaaagctgttggcagccttaaagggacgctggaggtcaccagtcgctggcttcccgaagacgtggatcttaaccagattcgaaagcgccttcagcggatgatcgactggatcagaagagcgttggtgtacttgaagtacagatgcatgacggccatcatgccaacgcccatgaactggctcttaatgagctggcggaactggcccttatcgtactccatgttggtagttgtgacaggacgaggctcctcgccgcttccaagcggagcaggctcgacgtatttcagtgtcgaaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgcggccgcaattaaccctcactaaaggatccctatagtgagtcgtattatgcggccgcgaattctcatgtttgaccgcttatcatcgataagctctgctttttgttgacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacaaggcggatcgccggaaaggacccgcaaatgataataattatcaattgcatactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgcaggataccaaggctgatgttgtagatattggtcaccggctgagggttttcgattgccgctgcgtggatagcaccatttgcgatcaggcngtccttgatgaatgacactccattgcgaataagttcgaaggagacggtgtcacgaatgcgctggtccagctcggtcgattgccttttgtgcagcagaggtatcaatctcaacgccaaggctcatcgaagcgcaatattgctgctcaccaaaacgcgtattgaccaggtgttcaacggcaaatttctgcccttctgatgtcagaaaggcaaagtgattttctttctggtattcagttgctgtgtgtcggtttcagcaaaaccaagctcgcgcaattcggctgtgcagatttagaaggcagatcaccagacagcaacggccaacggaaaacagcgcatacagaacatccgtcgccgcgccgacaacgtgataatttttatgacccatgatttatttccttttagacgtgagcctgtcgcacagcaaagccgccgaaagttcctcgaagctagcttcagacgtgtctagatacgtctgctttttgttgacttccattgttcattccacggacaaaaacagagaaaggaaacgacagaggccaaaaagctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacaaggcggatcgccggaaaggacccgcaaatgataataattatcaattgcatactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgcaggataccaaggctgatgttgtagatattggtcaccggctgagggttttcgattgccgctgcgtggatagcaccatttgcgatcaggcngtccttgatgaatgacactccattgcgaataagttcgaaggagacggtgtcacgaatgcgctggtccagctcggtcgattgccttttgtgcagcagaggtatcaatctcaacgccaaggctcatcgaagcgcaatattgctgctcaccaaaacgcgtattgaccaggtgttcaacggcaaatttctgcccttctgatgtcagaaaggcaaagtgattttctttctggtattcagttgctgtgtgtcggtttcagcaaaaccaagctcgcgcaattcggctgtgcagatttagaaggcagatcaccagacagcaacggccaacggaaaacagcgcatacagaacatccgtcgccgcgccgacaacgtgataatttttatgacccatgatttatttccttttagacgtgagcctgtcgcacagcaaagccgccgaaagttcctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctgcagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaa

SEQ ID NO: 5 - DNA, 21 bp, Artificial Sequence
  Primer
cgatgtaggagggcgtggata

SEQ ID NO: 6 - DNA, 21 bp, Artificial Sequence
  Primer
gcttctgcgggcgatttgtgt

SEQ ID NO: 7 - DNA, 20 bp, Artificial Sequence
  Primer
tgagaatatcaccggaattg

SEQ ID NO: 8 - DNA, 21 bp, Artificial Sequence
  Primer
agctcgacatactgttcttcc

SEQ ID NO: 9 - DNA, 24 bp, Artificial Sequence
  Primer
gtgaatggaaatcccatcgctgtc

SEQ ID NO: 10 - DNA, 24 bp, Artificial Sequence
  Primer
agtgggtactctaaaggccatacc

SEQ ID NO: 11 - DNA, 1771 bp, Haematococcus pluvialis
  CDS (166)..(1155)
ggcacgagcttgcacgcaagtcagcgcgcgcaagtcaacacctgccggtccacagcctcaaataataaagagctcaagcgtttgtgcgcctcgacgtggccagtctgcactgccttgaacccgcgagtctcccgccgcactgactgccatagcacagctagacgaatgcagctagcagcgacagtaatgttggagcagcttaccggaagcgctgaggcactcaaggagaaggagaaggaggttgcaggcagctctgacgtgttgcgtacatgggcgacccagtactcgcttccgtcagaagagtcagacgcggcccgcccgggactgaagaatgcctacaagccaccaccttccgacacaaagggcatcacaatggcgctacgtgtcatcggctcctgggccgcagtgttcctccacgccatttttcaaatcaagcttccgacctccttggaccagctgcactggctgcccgtgtcagatgccacagctcagctggttagcggcacgagcagcctgctcgacatcgtcgtagtattctttgtcctggagttcctgtacacaggcctttttatcaccacgcatgatgctatgcatggcaccatcgccatgagaaacaggcagcttaatgacttcttgggcagagtatgcatctccttgtacgcctggtttgattacaacatgctgcaccgcaagcattgggagcaccacaaccacactggcgaggtgggcaaggaccctgacttccacaggggaaaccctggcattgtgccctggtttgccagcttcatgtccagctacatgtcgatgtggcagtttgcgcgcctcgcatggtggacggtggtcatgcagctgctgggtgcgccaatggcgaacctgctggtgttcatggcggccgcgcccatcctgtccgccttccgcttgttctactttggcacgtacatgccccacaagcctgagcctggcgccgcgtcaggctcttcaccagccgtcatgaactggtggaagtcgcgcactagccaggcgtccgacctggtcagctttctgacctgctaccacttcgacctgcactgggagcaccaccgctggcccttcgccccctggtgggagctgcccaactgccgccgcctgtctggccgaggtctggttcctgcctagctggacacactgcagtgggccctgctgccagctgggcatgcaggttgtggcaggactgggtgaggtgaaaagctgcaggcgctgctgccggacacgctgcatgggctaccctgtgtagctgccgccactaggggagggggtttgtagctgtcgagcttgccccatggatgaagctgtgtagtggtgcagggagtacacccacaggccaacacccttgcaggagatgtcttgcgtcgggaggagtgttgggcagtgtagatgctatgattgtatcttaatgctgaagcctttaggggagcgacacttagtgctgggcaggcaacgccctgcaaggtgcaggcacaagctaggctggacgaggactcggtggcaggcaggtgaagaggtgcgggagggtggtgccacacccactgggcaagaccatgctgcaatgctggcggtgtggcagtgagagctgcgtgattaactgggctatggattgtttgagcagtctcacttattctttgatatagatactggtcaggcaggtcaggagagtgagtatgaacaagttgagaggtggtgcgctgcccctgcgcttatgaagctgtaacaataaagtggttcaaaaaaaaaa

SEQ ID NO: 12 - protein, 329 aa, Haematococcus pluvialis
MQLAATVMLEQLTGSAEALKEKEKEVAGSSDVLRTWATQYSLPSEESDAARPGLKNAYKPPPSDTKGITMALRVIGSWAAVFLHAIFQIKLPTSLDQLHWLPVSDATAQLVSGTSSLLDIVVVFFVLEFLYTGLFITTHDAMHGTIAMRNRQLNDFLGRVCISLYAWFDYNMLHRKHWEHHNHTGEVGKDPDFHRGNPGIVPWFASFMSSYMSMWQFARLAWWTVVMQLLGAPMANLLVFMAAAPILSAFRLFYFGTYMPHKPEPGAASGSSPAVMNWWKSRTSQASDLVSFLTCYHFDLHWEHHRWPFAPWWELPNCRRLSGRGLVPA

SEQ ID NO: 13 - DNA, 1662 bp, Haematococcus pluvialis
  CDS (168)..(1130)
cggggcaactcaagaaattcaacagctgcaagcgcgccccagcctcacagcgccaagtgagctatcgacgtggttgtgagcgctcgacgtggtccactgacgggcctgtgagcctctgcgctccgtcctctgccaaatctcgcgtcggggcctgcctaagtcgaagaatgcacgtcgcatcggcactaatggtcgagcagaaaggcagtgaggcagctgcttccagcccagacgtcttgagagcgtgggcgacacagtatcacatgccatccgagtcgtcagacgcagctcgtcctgcgctaaagcacgcctacaaacctccagcatctgacgccaagggcatcacgatggcgctgaccatcattggcacctggaccgcagtgtttttacacgcaatatttcaaatcaggctaccgacatccatggaccagcttcactggttgcctgtgtccgaagccacagcccagcttttgggcggaagcagcagcctactgcacatcgctgcagtcttcattgtacttgagttcctgtacactggtctattcatcaccacacatgacgcaatgcatggcaccatagctttgaggcacaggcagctcaatgatctccttggcaacatctgcatatcactgtacgcctggtttgactacagcatgctgcatcgcaagcactgggagcaccacaaccatactggcgaagtggggaaagaccctgacttccacaagggaaatcccggccttgtcccctggttcgccagcttcatgtccagctacatgtccctgtggcagtttgcccggctggcatggtgggcagtggtgatgcaaatgctgggggcgcccatggcaaatctcctagtcttcatggctgcagccccaatcttgtcagcattccgcctcttctacttcggcacttacctgccacacaagcctgagccaggccctgcagcaggctctcaggtgatggcctggttcagggccaagacaagtgaggcatctgatgtgatgagtttcctgacatgctaccactttgacctgcactgggagcaccacaggtggccctttgccccctggtggcagctgccccactgccgccgcctgtccgggcgtggcctggtgcctgccttggcatgacctggtccctccgctggtgacccagcgtctgcacaagagtgtcatgctacagggtgctgcggccagtggcagcgcagtgcactctcagcctgtatggggctaccgctgtgccactgagcactgggcatgccactgagcactgggcgtgctactgagcaatgggcgtgctactgagcaatgggcgtgctactgacaatgggcgtgctactggggtctggcagtggctaggatggagtttgatgcattcagtagcggtggccaacgtcatgtggatggtggaagtgctgaggggtttaggcagccggcatttgagagggctaagttataaatcgcatgctgctcatgcgcacatatctgcacacagccagggaaatcccttcgagagtgattatgggacacttgtattggtttcgtgctattgttttattcagcagcagtacttagtgagggtgagagcagggtggtgagagtggagtgagtgagtatgaacctggtcagcgaggtgaacagcctgtaatgaatgactctgtct

SEQ ID NO: 14 - protein, 320 aa, Haematococcus pluvialis
MHVASALMVEQKGSEAAASSPDVLRAWATQYHMPSESSDAARPALKHAYKPPASDAKGITMALTIIGTWTAVFLHAIFQIRLPTSMDQLHWLPVSEATAQLLGGSSSLLHIAAVFIVLEFLYTGLFITTHDAMHGTIALRHRQLNDLLGNICISLYAWFDYSMLHRKHWEHHNHTGEVGKDPDFHKGNPGLVPWFASFMSSYMSLWQFARLAWWAVVMQMLGAPMANLLVFMAAAPILSAFRLFYFGTYLPHKPEPGPAAGSQVMAWFRAKTSEASDVMSFLTCYHFDLHWEHHRWPFAPWWQLPHCRRLSGRGLVPALA

SEQ ID NO: 15 - DNA, 729 bp, Agrobacterium aurantiacum
  CDS (1)..(729)
atgagcgcacatgccctgcccaaggcagatctgaccgccaccagcctgatcgtctcgggcggcatcatcgccgcttggctggccctgcatgtgcatgcgctgtggtttctggacgcagcggcgcatcccatcctggcgatcgcaaatttcctggggctgacctggctgtcggtcggattgttcatcatcgcgcatgacgcgatgcacgggtcggtggtgccggggcgtccgcgcgccaatgcggcgatgggccagcttgtcctgtggctgtatgccggattttcgtggcgcaagatgatcgtcaagcacatggcccatcaccgccatgccggaaccgacgacgaccccgatttcgaccatggcggcccggtccgctggtacgcccgcttcatcggcacctatttcggctggcgcgaggggctgctgctgcccgtcatcgtgacggtctatgcgctgatccttggggatcgctggatgtacgtggtcttctggccgctgccgtcgatcctggcgtcgatccagctgttcgtgttcggcacctggctgccgcaccgccccggccacgacgcgttcccggaccgccacaatgcgcggtcgtcgcggatcagcgaccccgtgtcgctgctgacctgctttcactttggcggttatcatcacgaacaccacctgcacccgacggtgccgtggtggcgcctgcccagcacccgcaccaagggggacaccgcatga

SEQ ID NO: 16 - protein, 242 aa, Agrobacterium aurantiacum
MSAHALPKADLTATSLIVSGGIIAAWLALHVHALWFLDAAAHPILAIANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 17 - DNA, 1631 bp, Alcaligenes sp.
  CDS (99)..(827)
ctgcaggccgggcccggtggccaatggtcgcaaccggcaggactggaacaggacggcgggccggtctaggctgtcgccctacgcagcaggagtttcggatgtccggacggaagcctggcacaactggcgacacgatcgtcaatctcggtctgaccgccgcgatcctgctgtgctggctggtcctgcacgcctttacgctatggttgctagatgcggccgcgcatccgctgcttgccgtgctgtgcctggctgggctgacctggctgtcggtcgggctgttcatcatcgcgcatgacgcaatgcacgggtccgtggtgccggggcggccgcgcgccaatgcggcgatcgggcaactggcgctgtggctctatgcggggttctcgtggcccaagctgatcgccaagcacatgacgcatcaccggcacgccggcaccgacaacgatcccgatttcggtcacggagggcccgtgcgctggtacggcagcttcgtctccacctatttcggctggcgagagggactgctgctaccggtgatcgtcaccacctatgcgctgatcctgggcgatcgctggatgtatgtcatcttctggccggtcccggccgttctggcgtcgatccagattttcgtcttcggaacttggctgccccaccgcccgggacatgacgattttcccgaccggcacaacgcgaggtcgaccggcatcggcgacccgttgtcactactgacctgcttccatttcggcggctatcaccacgaacatcacctgcatccgcatgtgccgtggtggcgcctgcctcgtacacgcaagaccggaggccgcgcatgacgcaattcctcattgtcgtggcgacagtcctcgtgatggagctgaccgcctattccgtccaccgctggattatgcacggccccctaggctggggctggcacaagtcccatcacgaagagcacgaccacgcgttggagaagaacgacctctacggcgtcgtcttcgcggtgctggcgacgatcctcttcaccgtgggcgcctattggtggccggtgctgtggtggatcgccctgggcatgacggtctatgggttgatctatttcatcctgcacgacgggcttgtgcatcaacgctggccgtttcggtatattccgcggcggggctatttccgcaggctctaccaagctcatcgcctgcaccacgcggtcgaggggcgggaccactgcgtcagcttcggcttcatctatgccccacccgtggacaagctgaagcaggatctgaagcggtcgggtgtcctgcgcccccaggacgagcgtccgtcgtgatctctgatcccggcgtggccgcatgaaatccgacgtgctgctggcaggggccggccttgccaacggactgatcgcgctggcgatccgcaaggcgcggcccgaccttcgcgtgctgctgctggaccgtgcggcgggcgcctcggacgggcatacttggtcctgccacgacaccgatttggcgccgcactggctggaccgcctgaagccgatcaggcgtggcgactggcccgatcaggaggtgcggttcccagaccattcgcgaaggctccgggccggatatggctcgatcgacgggcgggggctgatgcgtgcggtgacc

SEQ ID NO: 18 - protein, 242 aa, Alcaligenes sp.
MSGRKPGTTGDTIVNLGLTAAILLCWLVLHAFTLWLLDAAAHPLLAVLCLAGLTWLSVGLFIIAHDAMHGSVVPGRPRANAAIGQLALWLYAGFSWPKLIAKHMTHHRHAGTDNDPDFGHGGPVRWYGSFVSTYFGWREGLLLPVIVTTYALILGDRWMYVIFWPVPAVLASIQIFVFGTWLPHRPGHDDFPDRHNARSTGIGDPLSLLTCFHFGGYHHEHHLHPHVPWWRLPRTRKTGGRA

SEQ ID NO: 19 - DNA, 729 bp, Paracoccus marcusii
  CDS (1)..(729)
atgagcgcacatgccctgcccaaggcagatctgaccgccacaagcctgatcgtctcgggcggcatcatcgccgcatggctggccctgcatgtgcatgcgctgtggtttctggacgcggcggcccatcccatcctggcggtcgcgaatttcctggggctgacctggctgtcggtcggattgttcatcatcgcgcatgacgcgatgcacgggtcggtcgtgccggggcgtccgcgcgccaatgcggcgatgggccagcttgtcctgtggctgtatgccggattttcgtggcgcaagatgatcgtcaagcacatggcccatcaccgccatgccggaaccgacgacgacccagatttcgaccatggcggcccggtccgctggtacgcccgcttcatcggcacctatttcggctggcgcgaggggctgctgctgcccgtcatcgtgacggtctatgcgctgatcctgggggatcgctggatgtacgtggtcttctggccgttgccgtcgatcctggcgtcgatccagctgttcgtgttcggcacttggctgccgcaccgccccggccacgacgcgttcccggaccgccataatgcgcggtcgtcgcggatcagcgaccctgtgtcgctgctgacctgctttcattttggcggttatcatcacgaacaccacctgcacccgacggtgccgtggtggcgcctgcccagcacccgcaccaagggggacaccgcatga

SEQ ID NO: 20 - protein, 242 aa, Paracoccus marcusii
MSAHALPKADLTATSLIVSGGIIAAWLALHVHALWFLDAAAHPILAVANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 21 - DNA, 1629 bp, Synechocystis sp.
  CDS (1)..(1629)
atgatcaccaccgatgttgtcattattggggcggggcacaatggcttagtctgtgcagcctatttgctccaacggggcttgggggtgacgttactagaaaagcgggaagtaccagggggggcggccaccacagaagctctcatgccggagctatccccccagtttcgctttaaccgctgtgccattgaccacgaatttatctttctggggccggtgttgcaggagctaaatttagcccagtatggtttggaatatttattttgtgaccccagtgttttttgtccggggctggatggccaagcttttatgagctaccgttccctagaaaaaacctgtgcccacattgccacctatagcccccgagatgcggaaaaatatcggcaatttgtcaattattggacggatttgctcaacgctgtccagcctgcttttaatgctccgccccaggctttactagatttagccctgaactatggttgggaaaacttaaaatccgtgctggcgatcgccgggtcgaaaaccaaggcgttggattttatccgcactatgatcggctccccggaagatgtgctcaatgaatggttcgacagcgaacgggttaaagctcctttagctagactatgttcggaaattggcgctcccccatcccaaaagggtagtagctccggcatgatgatggtggccatgcggcatttggagggaattgccagaccaaaaggaggcactggagccctcacagaagccttggtgaagttagtgcaagcccaagggggaaaaatcctcactgaccaaaccgtcaaacgggtattggtggaaaacaaccaggcgatcggggtggaggtagctaacggagaacagtaccgggccaaaaaaggcgtgatttctaacatcgatgcccgccgtttatttttgcaattggtggaaccgggggccctagccaaggtgaatcaaaacctaggggaacgactggaacggcgcactgtgaacaataacgaagccattttaaaaatcgattgtgccctctccggtttaccccacttcactgccatggccgggccggaggatctaacgggaactattttgattgccgactcggtacgccatgtcgaggaagcccacgccctcattgccttggggcaaattcccgatgctaatccgtctttatatttggatattcccactgtattggaccccaccatggccccccctgggcagcacaccctctggatcgaattttttgccccctaccgcatcgccgggttggaagggacagggttaatgggcacaggttggaccgatgagttaaaggaaaaagtggcggatcgggtgattgataaattaacggactatgcccctaacctaaaatctctgatcattggtcgccgagtggaaagtcccgccgaactggcccaacggctgggaagttacaacggcaatgtctatcatctggatatgagtttggaccaaatgatgttcctccggcctctaccggaaattgccaactaccaaacccccatcaaaaatctttacttaacaggggcgggtacccatcccggtggctccatatcaggtatgcccggtagaaattgcgctcgggtctttttaaaacaacaacgtcgtttttggtaa

SEQ ID NO: 22 - protein, 542 aa, Synechocystis sp.
MITTDVVIIGAGHNGLVCAAYLLQRGLGVTLLEKREVPGGAATTEALMPELSPQFRFNRCAIDHEFIFLGPVLQELNLAQYGLEYLFCDPSVFCPGLDGQAFMSYRSLEKTCAHIATYSPRDAEKYRQFVNYWTDLLNAVQPAFNAPPQALLDLALNYGWENLKSVLAIAGSKTKALDFIRTMIGSPEDVLNEWFDSERVKAPLARLCSEIGAPPSQKGSSSGMMMVAMRHLEGIARPKGGTGALTEALVKLVQAQGGKILTDQTVKRVLVENNQAIGVEVANGEQYRAKKGVISNIDARRLFLQLVEPGALAKVNQNLGERLERRTVNNNEAILKIDCALSGLPHFTAMAGPEDLTGTILIADSVRHVEEAHALIALGQIPDANPSLYLDIPTVLDPTMAPPGQHTLWIEFFAPYRIAGLEGTGLMGTGWTDELKEKVADRVIDKLTDYAPNLKSLIIGRRVESPAELAQRLGSYNGNVYHLDMSLDQMMFLRPLPEIANYQTPIKNLYLTGAGTHPGGSISGMPGRNCARVFLKQQRRFW

SEQ ID NO: 23 - DNA, 776 bp, Bradyrhizobium sp.
  CDS (1)..(774)
atgcatgcagcaaccgccaaggctactgagttcggggcctctcggcgcgacgatgcgaggcagcgccgcgtcggtctcacgctggccgcggtcatcatcgccgcctggctggtgctgcatgtcggtctgatgttcttctggccgctgacccttcacagcctgctgccggctttgcctctggtggtgctgcagacctggctctatgtaggcctgttcatcatcgcgcatgactgcatgcacggctcgctggtgccgttcaagccgcaggtcaaccgccgtatcggacagctctgcctgttcctctatgccgggttctccttcgacgctctcaatgtcgagcaccacaagcatcaccgccatcccggcacggccgaggatcccgatttcgacgaggtgccgccgcacggcttctggcactggttcgccagctttttcctgcactatttcggctggaagcaggtcgcgatcatcgcagccgtctcgctggtttatcagctcgtcttcgccgttcccttgcagaacatcctgctgttctgggcgctgcccgggctgctgtcggcgctgcagctgttcaccttcggcacctatctgccgcacaagccggccacgcagcccttcgccgatcgccacaacgcgcggacgagcgaatttcccgcgtggctgtcgctgctgacctgcttccacttcggctttcatcacgagcatcatctgcatcccgatgcgccgtggtggcggctgccggagatcaagcggcgggccctggaaaggcgtgacta

SEQ ID NO: 24 - protein, 258 aa, Bradyrhizobium sp.
MHAATAKATEFGASRRDDARQRRVGLTLAAVIIAAWLVLHVGLMFFWPLTLHSLLPALPLVVLQTWLYVGLFIIAHDCMHGSLVPFKPQVNRRIGQLCLFLYAGFSFDALNVEHHKHHRHPGTAEDPDFDEVPPHGFWHWFASFFLHYFGWKQVAIIAAVSLVYQLVFAVPLQNILLFWALPGLLSALQLFTFGTYLPHKPATQPFADRHNARTSEFPAWLSLLTCFHFGFHHEHHLHPDAPWWRLPEIKRRALERRD

SEQ ID NO: 25 - DNA, 777 bp, Nostoc sp.
  CDS (1)..(777)
atggttcagtgtcaaccatcatctctgcattcagaaaaactggtgttattgtcatcgacaatcagagatgataaaaatattaataagggtatatttattgcctgctttatcttatttttatgggcaattagtttaatcttattactctcaatagatacatccataattcataagagcttattaggtatagccatgctttggcagaccttcttatatacaggtttatttattactgctcatgatgccatgcacggcgtagtttatcccaaaaatcccagaataaataattttataggtaagctcactctaatcttgtatggactactcccttataaagatttattgaaaaaacattggttacaccacggacatcctggtactgatttagaccctgattattacaatggtcatccccaaaacttctttctttggtatctacattttatgaagtcttattggcgatggacgcaaattttcggattagtgatgatttttcatggacttaaaaatctggtgcatataccagaaaataatttaattatattttggatgataccttctattttaagttcagtacaactattttattttggtacatttttgcctcataaaaagctagaaggtggttatactaacccccattgtgcgcgcagtatcccattacctcttttttggtcttttgttacttgttatcacttcggctaccacaaggaacatcacgaataccctcaacttccttggtggaaattacctgaagctcacaaaatatctttataa

SEQ ID NO: 26 - protein, 258 aa, Nostoc sp.
MVQCQPSSLHSEKLVLLSSTIRDDKNINKGIFIACFILFLWAISLILLLSIDTSIIHKSLLGIAMLWQTFLYTGLFITAHDAMHGVVYPKNPRINNFIGKLTLILYGLLPYKDLLKKHWLHHGHPGTDLDPDYYNGHPQNFFLWYLHFMKSYWRWTQIFGLVMIFHGLKNLVHIPENNLIIFWMIPSILSSVQLFYFGTFLPHKKLEGGYTNPHCARSIPLPLFWSFVTCYHFGYHKEHHEYPQLPWWKLPEAHKISL

SEQ ID NO: 27 - DNA, 789 bp, Nostoc punctiforme
  CDS (1)..(789)
ttgaatttttgtgataaaccagttagctattatgttgcaatagagcaattaagtgctaaagaagatactgtttgggggctggtgattgtcatagtaattattagtctttgggtagctagtttggcttttttactagctattaattatgccaaagtcccaatttggttgatacctattgcaatagtttggcaaatgttcctttatacagggctatttattactgcacatgatgctatgcatgggtcagtttatcgtaaaaatcccaaaattaataattttatcggttcactagctgtagcgctttacgctgtgtttccatatcaacagatgttaaagaatcattgcttacatcatcgtcatcctgctagcgaagttgacccagattttcatgatggtaagagaacaaacgctattttctggtatctccatttcatgatagaatactccagttggcaacagttaatagtactaactatcctatttaatttagctaaatacgttttgcacatccatcaaataaatctcatcttattttggagtattcctccaattttaagttccattcaactgttttatttcggaacatttttgcctcatcgagaacccaagaaaggatatgtttatccccattgcagccaaacaataaaattgccaacttttttgtcatttatcgcttgctaccactttggttatcatgaagaacatcatgagtatccccatgtaccttggtggcaacttccatctgtatataagcagagagtattcaacaattcagtaaccaattcgtaa

SEQ ID NO: 28 - protein, 262 aa, Nostoc punctiforme
LNFCDKPVSYYVAIEQLSAKEDTVWGLVIVIVIISLWVASLAFLLAINYAKVPIWLIPIAIVWQMFLYTGLFITAHDAMHGSVYRKNPKINNFIGSLAVALYAVFPYQQMLKNHCLHHRHPASEVDPDFHDGKRTNAIFWYLHFMIEYSSWQQLIVLTILFNLAKYVLHIHQINLILFWSIPPILSSIQLFYFGTFLPHREPKKGYVYPHCSQTIKLPTFLSFIACYHFGYHEEHHEYPHVPWWQLPSVYKQRVFNNSVTNS

SEQ ID NO: 29 - DNA, 762 bp, Nostoc punctiforme
  CDS (1)..(762)
gtgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatag

SEQ ID NO: 30 - protein, 253 aa, Nostoc punctiforme
VIQLEQPLSHQAKLTPVLRSKSQFKGLFIAIVIVSAWVISLSLLLSLDISKLKFWMLLPVILWQTFLYTGLFITSHDAMHGVVFPQNTKINHLIGTLTLSLYGLLPYQKLLKKHWLHHHNPASSIDPDFHNGKHQSFFAWYFHFMKGYWSWGQIIALTIIYNFAKYILHIPSDNLTYFWVLPSLLSSLQLFYFGTFLPHSEPIGGYVQPHCAQTISRPIWWSFITCYHFGYHEEHHEYPHISWWQLPEIYKAK

SEQ ID NO: 31 - DNA, 1608 bp, Haematococcus pluvialis
  CDS (3)..(971)
ctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaaaaaaaaaaaaaaaaaaaaaaa

SEQ ID NO: 32 - protein, 322 aa, Haematococcus pluvialis
TFHKPVSGASALPHIGPPPHLHRSFAATTMLSKLQSISVKARRVELARDITRPKVCLHAQRCSLVRLRVAAPQTEEALGTVQAAGAGDEHSADVALQQLDRAIAERRARRKREQLSYQAAAIAASIGVSGIAIFATYLRFAMHMTVGGAVPWGEVAGTLLLVVGGALGMEMYARYAHKAIWHESPLGWLLHKSHHTPRTGPFEANDLFAIINGLPAMLLCTFGFWLPNVLGAACFGAGLGITLYGMAYMFVHDGLVHRRFPTGPIAGLPYMKRLTVAHQLHHSGKYGGAPWGMFLGPQELQHIPGAAEEVERLVLELDWSKR

SEQ ID NO: 33 - DNA, 528 bp, Erwinia uredovora
  CDS (1)..(528)
atgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaa

SEQ ID NO: 34 - protein, 175 aa, Erwinia uredovora
MLWIWNALIVFVTVIGMEVIAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLQATLRERHGARAGAARDAQGGEDEPASGK

SEQ ID NO: 35 - DNA, 1520 bp, Artificial Sequence
  Promoter
ctcgagtaccgaggcggaacggcaggaatgtttccctctcttttagagggcaattctttatccaatgtcatgttgatgctagatatttctgtctcttataataaggcgaatacccatttttgaattgaagttgagataaaaaaaaagggggcccaatttgtcaacgccaaagagtcaagctttttctttggctttagccgaacaatctaagacttattgtttttgaagatatttgaccttttctagatattccttcaagtaaagcttttttcgagttttttttttttttctttgtgaaggatttattgttattggtatccattttttattggaagacaagataagttaatattgattttgcttaaagattaaaaggaaatcagaaaacgacaataaaaaatgtaacggacaaactatggtgtcgattataagtctaaatccttaaaaaatgacaacgagttgctttcctctgaaaacaattcttttgtctttgcaagaaaggtttcttttttgtttgcttgcattacttaaacatcaaatcaaatgaaaggaataaagcagatttgagggcgaataaggattttctggtcaacaagatgtgagtgacacctaaggaactaaatgccattcatttgttttaaaacgacatcaaagattgatgatcaacaggattgagagagagaaaaagaactcgtgtcatttatttctgttgactgaaattttatatttagaaaaaatgtcaaatctatagctttagctatattacataacatttgaaataataataataaaaaaagacacattagagacacttttcaaactctaaataactgtctataaacacaaagaaaacaaagacctctataacaacttattagatttttctcgtacttttgtctaaagatgatgtattcttgttatcccacacttctttcatttgttcttgatgctactaaatatacaaaatttcttttttgcaagagatattattccaaaaattttcaaaaagaaatttttttcacaatagcagttgatcgtgtaacccaaagaggttctttgttattttgcacttccgctttgcggtgatgcatattcaaagtaatatatggaataaacaacgtgtttaagcatgaaagaaaggaaacaaaggccgctttgaacaaatgcataatatttcagacaaaaatgatctaaagcaagcagtaaatcaaacaagaaacattgctgattcgcgttagaaaacgataaaagtctaataagccactaagtatacttcaatgaactttttgtatgcttatggtccaatcagaccaataatttgtgaccattcctgaggtggctttggtgatgcggaaacagaaaaaaattttctcaccaatcgatttaaaaaacaatttctgctttgaaccaaaactttttttttctctttaatcattaactttatcaagtatgtacctaccctcaaagtcctcactcaagcacaattatgctaacattgttccaccttctctttagaaatg

SEQ ID NO: 36 - DNA, 16245 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttaatctatacaatgctccatagactcacattgatattgtcgaagatttcgatgctgacttagtagagcaactacaaaagttagcagagaagcatgatttcttaatctttgaagaccgcaagtttgcagatatcggtatgtgaattctatctattttttttctgatgtgtgcatggatgactcatgatcatattcttaggtaatactgtcaagcatcaatatggcaagggcgtttacaagattgcttcttggtctcatattactaatgctcacacagttcctggagaaggtattatcaagggacttgccgaagtcggcctccctcttggtcgtggcttgcttttgctagcagaaatgtcatctcaaggtgcattaactaagggtatttacactgccgaatctgtcaatatggctcgccgcaacaaagatttcgtttttggctttattgcacaacacaaaatgaatcagtatgatgatgaggattttgttgtcatgtcgcctgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 37 - DNA, 17877 bp, Artificial Sequence
  Promoter
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttttttcgagttttttttttttttctttgtgaaggatttattgttattggtatccattttttattggaagacaagataagttaatattgattttgcttaaagattaaaaggaaatcagaaaacgacaataaaaaatgtaacggacaaactatggtgtcgattataagtctaaatccttaaaaaatgacaacgagttgctttcctctgaaaacaattcttttgtctttgcaagaaaggtttcttttttgtttgcttgcattacttaaacatcaaatcaaatgaaaggaataaagcagatttgagggcgaataaggattttctggtcaacaagatgtgagtgacacctaaggaactaaatgccattcatttgttttaaaacgacatcaaagattgatgatcaacaggattgagagagagaaaaagaactcgtgtcatttatttctgttgactgaaattttatatttagaaaaaatgtcaaatctatagctttagctatattacataacatttgaaataataataataaaaaaagacacattagagacacttttcaaactctaaataactgtctataaacacaaagaaaacaaagacctctataacaacttattagatttttctcgtacttttgtctaaagatgatgtattcttgttatcccacacttctttcatttgttcttgatgctactaaatatacaaaatttcttttttgcaagagatattattccaaaaattttcaaaaagaaatttttttcacaatagcagttgatcgtgtaacccaaagaggttctttgttattttgcacttccgctttgcggtgatgcatattcaaagtaatatatggaataaacaacgtgtttaagcatgaaagaaaggaaacaaaggccgctttgaacaaatgcataatatttcagacaaaaatgatctaaagcaagcagtaaatcaaacaagaaacattgctgattcgcgttagaaaacgataaaagtctaataagccactaagtatacttcaatgaactttttgtatgcttatggtccaatcagaccaataatttgtgaccattcctgaggtggctttggtgatgcggaaacagaaaaaaattttctcaccaatcgatttaaaaaacaatttctgctttgaaccaaaactttttttttctctttaatcattaactttatcaagtatgtacctaccctcaaagtcctcactcaagcacaattatgctaacattgttccaccttctctttagaaatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 38 - DNA, 17238 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttctaccgcttggaccagtccagttccaggaccagtcgctccacctcctccgccgcacctggaatgtgctgcagctcctgtggacccaagaacataccccagggcgcgccaccgtacttgccgctgtggtgtagctggtgggccactgtcaggcgcttcatgtagggcaggccagcgatgggcccggtgggaaagcgcctgtgcaccaggccatcgtgtacaaacatatatgccatgccgtatagcgtgatgcccagccccgctccaaagcaggccgcccccaggacgttgggcagccagaagccaaaggtacacaggagcatggcgggcagtccattgatgattgcaaacaagtcgttggcttcaaagggtccagtgcgaggtgtgtggtggctcttgtgcagcagccagcccagaggcgactcatgccagatggctttgtgtgcatagcgggcatacatctccatgccgagcgcgccaccaaccaccaagaggagagtgccagccacttcaccccatggcactgcgccgcccacggtcatgtgcatggcaaatctcaggtaggtggcgaagatggcaatgcctgacacgccaattgatgctgcaatggcggcagcctggtatgacagctgctcccgtttgcgccgggcacgacgctctgcgatagcccggtcaagctgctggagtgctacatcggcgctgtgctcatcgcccgcgccggcagcctgcacggttcccagcgcctcctctgtctgtggtgctgccactcgcagccgaactaacgagcaccgctgagcatgcaggcagactttgggccgcgtgatgtcgcgggctagttcaacgcggcgggccttgacgctgattgactgcagcttcgacagcatagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 39 - DNA, 17238 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 40 - DNA, 18449 bp, Artificial Sequence
  Plasmid
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgaattcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctagcccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 41 - DNA, 18449 bp, Artificial Sequence
  Plasmid
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcgggcgattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgaattcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctagcccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 42 - DNA, 17593 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttttttcgagttttttttttttttctttgtgaaggatttattgttattggtatccattttttattggaagacaagataagttaatattgattttgcttaaagattaaaaggaaatcagaaaacgacaataaaaaatgtaacggacaaactatggtgtcgattataagtctaaatccttaaaaaatgacaacgagttgctttcctctgaaaacaattcttttgtctttgcaagaaaggtttcttttttgtttgcttgcattacttaaacatcaaatcaaatgaaaggaataaagcagatttgagggcgaataaggattttctggtcaacaagatgtgagtgacacctaaggaactaaatgccattcatttgttttaaaacgacatcaaagattgatgatcaacaggattgagagagagaaaaagaactcgtgtcatttatttctgttgactgaaattttatatttagaaaaaatgtcaaatctatagctttagctatattacataacatttgaaataataataataaaaaaagacacattagagacacttttcaaactctaaataactgtctataaacacaaagaaaacaaagacctctataacaacttattagatttttctcgtacttttgtctaaagatgatgtattcttgttatcccacacttctttcatttgttcttgatgctactaaatatacaaaatttcttttttgcaagagatattattccaaaaattttcaaaaagaaatttttttcacaatagcagttgatcgtgtaacccaaagaggttctttgttattttgcacttccgctttgcggtgatgcatattcaaagtaatatatggaataaacaacgtgtttaagcatgaaagaaaggaaacaaaggccgctttgaacaaatgcataatatttcagacaaaaatgatctaaagcaagcagtaaatcaaacaagaaacattgctgattcgcgttagaaaacgataaaagtctaataagccactaagtatacttcaatgaactttttgtatgcttatggtccaatcagaccaataatttgtgaccattcctgaggtggctttggtgatgcggaaacagaaaaaaattttctcaccaatcgatttaaaaaacaatttctgctttgaaccaaaactttttttttctctttaatcattaactttatcaagtatgtacctaccctcaaagtcctcactcaagcacaattatgctaacattgttccaccttctctttagaaatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 43 - DNA, 16954 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 44 - DNA, 16954 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 45 - DNA, 19491 bp, Artificial Sequence
  Plasmid
agcttggtaccgagctcggatccactagtaacggccgccagtgtgctggaattcgcccttgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgca

SEQ ID NO: 46 - DNA, 21300 bp, Artificial Sequence
  Plasmid
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgtcaatactcacttatctggaatttcatctctactatacactacctgtccttgcggcattgtgttggctgctaaagccgtttcactcacagcaagacaatctcaagtataaatttttaatgttgatggccgcctctaccgcatcgatttgggacaattatatcgtttatcatcgcgcttggtggtactgtcctacttgtgttgtggctgtcattggctatgtacctctagaagaatacatgttctttatcatcatgactttaatgactgtcgcgttctcaaactttgttatgcgttggcacttgcatactttctttattagacccaacacttcttggaagcaaacactattagtacgccttgtgcctgtttcagctttattggcaatcacttatcatgcttggcacttgacactgccaaataaaccttcattttatggttcatgcatcctttggtatgcttgtcctgtgttggctattctttggctgggtgctggcgaatatatcttgcgtcgacctgtggctgtccttttgtctattgttatccctagtgtatacctatgttgggctgatatcgtcgctattagtgctggcacatggcatatttctcttagaacaagcactggcaaaatggtagtacccgatttacctgtagaagaatgcctgttttttactttgatcaacacagtcttggtttttgctacctgtgctatagaccgcgctcaggccatcctccatgtgagcgcgcgtaatacgactcactatagggcgaattggagctccaccgcggtggcggccgctctagaactagtggatcccccgggctgcaggaattcggcacgagctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcgggctcaggccatcctccatctgtacaaatcatctgttcaaaatcaaaaccctaaacaagccatttcccttttccagcatgtcaaagagctagcatgggccttctgtcttcctgaccaaatgctcaacaatgaattgtttgatgatcttactatcagctgggatattttacgtaaagcctcaaagtcattctatactgcatctgccgtttttccaagttatgtacgtcaagacttgggtgttctctatgctttctgcagagctaccgatgacctgtgcgatgatgaatccaaatctgttcaagaaagaagagaccaattagatcttactcgacaatttgttcgtgatctctttagccaaaagaccagtgcgcctattgtgattgattgggaattgtatcaaaaccaacttcctgcttcttgtatatcagcctttagagcctttactcgccttcgccatgtccttgaagtagaccctgtagaagaactattagatggttacaaatgggatcttgagcgtcgtcctatccttgatgaacaagacttggaggcatactctgcttgtgtggccagtagtgtgggtgaaatgtgcacacgtgtgattcttgctcaagaccaaaaggaaaatgatgcttggataattgaccgtgcacgtgagatggggctggtgctacaatacgttaacattgctcgagacattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgagttcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 47 - DNA, 17756 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttcattttgctttgtaaatttctggtaactgccaccaagaaatatgaggatattcgtgatgttcctcgtggtagccaaaatgatagcacgtgataaatgaccaccaaataggacggctaattgtttgggcacaatgaggctgaacataaccccctattggttcactatggggtaaaaaagtaccaaaatagaataattgtaatgaacttaaaagcgagggtagcacccaaaagtaagttagattatcacttgggatatggagtatgtatttagcaaagttataaataatagtcaacgcaattatttgcccccaactccagtaacctttcataaaatgaaaataccaagcaaagaaactttggtgtttaccattgtgaaaatccgggtctattgagcttgctggattgtggtggtgtaaccaatgttttttcaatagtttttgatatggtaaaagaccataaagggatagggtcaatgttccaatcaaatgattaatcttggtgttttggggaaatactacgccatgcatggcatcatgagatgtaataaataatcccgtatataaaaatgtttgccatagtataacaggcaataacatccaaaattttagctttgagatgtcaagggaaagtaataaactcaggctaatgacccatgcgctaacaatgacaatagcaatgaaaagccccttaaactgagatttacttctcagtactggagtcagttttgcttgatgactgagtggttgttctaactggatcatttctaaagagaaggtggaacaatgttagcataattgtgcttgagtgaggactttgagggtaggtacatacttgataaagttaatgattaaagagaaaaaaaaagttttggttcaaagcagaaattgttttttaaatcgattggtgagaaaatttttttctgtttccgcatcaccaaagccacctcaggaatggtcacaaattattggtctgattggaccataagcatacaaaaagttcattgaagtatacttagtggcttattagacttttatcgttttctaacgcgaatcagcaatgtttcttgtttgatttactgcttgctttagatcatttttgtctgaaatattatgcatttgttcaaagcggcctttgtttcctttctttcatgcttaaacacgttgtttattccatatattactttgaatatgcatcaccgcaaagcggaagtgcaaaataacaaagaacctctttgggttacacgatcaactgctattgtgaaaaaaatttctttttgaaaatttttggaataatatctcttgcaaaaaagaaattttgtatatttagtagcatcaagaacaaatgaaagaagtgtgggataacaagaatacatcatctttagacaaaagtacgagaaaaatctaataagttgttatagaggtctttgttttctttgtgtttatagacagttatttagagtttgaaaagtgtctctaatgtgtctttttttattattattatttcaaatgttatgtaatatagctaaagctatagatttgacattttttctaaatataaaatttcagtcaacagaaataaatgacacgagttctttttctctctctcaatcctgttgatcatcaatctttgatgtcgttttaaaacaaatgaatggcatttagttccttaggtgtcactcacatcttgttgaccagaaaatccttattcgccctcaaatctgctttattcctttcatttgatttgatgtttaagtaatgcaagcaaacaaaaaagaaacctttcttgcaaagacaaaagaattgttttcagaggaaagcaactcgttgtcattttttaaggatttagacttataatcgacaccatagtttgtccgttacattttttattgtcgttttctgatttccttttaatctttaagcaaaatcaatattaacttatcttgtcttccaataaaaaatggataccaataacaataaatccttcacaaagaaaaaaaaaaaaaactcgaaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 48 - DNA, 17118 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 49 - DNA, 18449 bp, Artificial Sequence; identical to SEQ ID NO: 41
  Plasmid

SEQ ID NO: 50 - DNA, 18617 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 51 - DNA, 18333 bp, Artificial Sequence
  Plasmid
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgttgtggatttggaatgccctgatcgttttcgttaccgtgattggcatggaagtgattgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaagggcctgaccagaggcggccagcagcagcgttaatttttcgggcgtggtcgttgactgccgctgatcccaaagcttgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatagaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 52 - DNA, 17 bp, Artificial Sequence
  Primer
gcngarggnathtggta

SEQ ID NO: 53 - DNA, 20 bp, Artificial Sequence
  Primer
tcngcnagraadatrttrtg

SEQ ID NO: 54 - DNA, 27 bp, Artificial Sequence
  Primer
aagtgacaccggttacacgcttgtctt

SEQ ID NO: 55 - DNA, 27 bp, Artificial Sequence
  Primer
gcttatcaccatctgttacctccttgc

SEQ ID NO: 56 - DNA, 32 bp, Artificial Sequence
  Primer
agagagggatccttaaatgcgaatatcgttgc

SEQ ID NO: 57 - DNA, 32 bp, Artificial Sequence
  Primer
agagagggatccatgtctgatcaaaagaagca

SEQ ID NO: 58 - DNA, 37 bp, Artificial Sequence
  Primer
actttattggatccttaaatgcgaatatcgttgctgc

SEQ ID NO: 59 - DNA, 38 bp, Artificial Sequence
  Primer
gttccaattggccacatgaagagtaagacaggaaacag

SEQ ID NO: 60 - DNA, 38 bp, Artificial Sequence
  Primer
cctgtcttactcttcatgtggccaattggaaccaacac

SEQ ID NO: 61 - DNA, 38 bp, Artificial Sequence
  Primer
ctattttaatcatatgtctgatcaaaagaagcatattg

SEQ ID NO: 62 - DNA, 16103 bp, Artificial Sequence
  Primer
gatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatcgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagctagaagtcctcgtgtactgtgtaagcgcccactccacatctccactcgacctgcaggcatgcaagcttgagtctatcgcctccaaaaagtacggtgctgaattcagatatcaatcgcctgttgctaaaattaacactgtcgataaagacaagcgtgtaaccggtgtcactttggaaagcggagaagtcattgaagccgatgcagtcgtatgtaatgcggatcttgtttatgcttatcaccatctgttacctccttgcaattggacaaagaagacattagcctcaaagaaactcacttcatcatctatttcgttttattggtccatgtcaacaaaggtgcctcaattagacgtacacaatatcttcttggctgaagcctacaaggaaagttttgatgagattttcaacgacttcggtttgccctctgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggg

SEQ ID NO: 63 - DNA, 25 bp, Artificial Sequence
  Primer
ggcgtacttgaaggaacccttaccg

SEQ ID NO: 64 - DNA, 25 bp, Artificial Sequence
  Primer
attgatgctcccggtcaccgtgatt

SEQ ID NO: 65 - DNA, 500 bp, Blakeslea trispora
aatctatacaatgctccatagactcacattgatattgtcgaagatttcgatgctgacttagtagagcaactacaaaagttagcagagaagcatgatttcttaatctttgaagaccgcaagtttgcagatatcggtatgtgaattctatctattttttttctgatgtgtgcatggatgactcatgatcatattcttaggtaatactgtcaagcatcaatatggcaagggcgtttacaagattgcttcttggtctcatattactaatgctcacacagttcctggagaaggtattatcaagggacttgccgaagtcggcctccctcttggtcgtggcttgcttttgctagcagaaatgtcatctcaaggtgcattaactaagggtatttacactgccgaatctgtcaatatggctcgccgcaacaaagatttcgtttttggctttattgcacaacacaaaatgaatcagtatgatgatgaggattttgttgtcatgtcgcctg

SEQ ID NO: 66 - DNA, 611 bp, Blakeslea trispora
gagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctct

SEQ ID NO: 67 - DNA, 720 bp, Blakeslea trispora
atgtcaatactcacttatctggaatttcatctctactatacactacctgtccttgcggcattgtgttggctgctaaagccgtttcactcacagcaagacaatctcaagtataaatttttaatgttgatggccgcctctaccgcatcgatttgggacaattatatcgtttatcatcgcgcttggtggtactgtcctacttgtgttgtggctgtcattggctatgtacctctagaagaatacatgttctttatcatcatgactttaatgactgtcgcgttctcaaactttgttatgcgttggcacttgcatactttctttattagacccaacacttcttggaagcaaacactattagtacgccttgtgcctgtttcagctttattggcaatcacttatcatgcttggcacttgacactgccaaataaaccttcattttatggttcatgcatcctttggtatgcttgtcctgtgttggctattctttggctgggtgctggcgaatatatcttgcgtcgacctgtggctgtccttttgtctattgttatccctagtgtatacctatgttgggctgatatcgtcgctattagtgctggcacatggcatatttctcttagaacaagcactggcaaaatggtagtacccgatttacctgtagaagaatgcctgttttttactttgatcaacacagtcttggtttttgctacctgtgctatagaccgc

SEQ ID NO: 68 - DNA, 1089 bp, Blakeslea trispora
ctgtacaaatcatctgttcaaaatcaaaaccctaaacaagccatttcccttttccagcatgtcaaagagctagcatgggccttctgtcttcctgaccaaatgctcaacaatgaattgtttgatgatcttactatcagctgggatattttacgtaaagcctcaaagtcattctatactgcatctgccgtttttccaagttatgtacgtcaagacttgggtgttctctatgctttctgcagagctaccgatgacctgtgcgatgatgaatccaaatctgttcaagaaagaagagaccaattagatcttactcgacaatttgttcgtgatctctttagccaaaagaccagtgcgcctattgtgattgattgggaattgtatcaaaaccaacttcctgcttcttgtatatcagcctttagagcctttactcgccttcgccatgtccttgaagtagaccctgtagaagaactattagatggttacaaatgggatcttgagcgtcgtcctatccttgatgaacaagacttggaggcatactctgcttgtgtggccagtagtgtgggtgaaatgtgcacacgtgtgattcttgctcaagaccaaaaggaaaatgatgcttggataattgaccgtgcacgtgagatggggctggtgctacaatacgttaacattgctcgagacattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattag

SEQ ID NO: 69 - DNA, 611 bp, Blakeslea trispora
agagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctc

SEQ ID NO: 70 - DNA, 882 bp, Haematococcus pluvialis
atgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtag

SEQ ID NO: 71 - DNA, 528 bp, Erwinia uredovora; identical to SEQ ID NO: 33

SEQ ID NO: 72 - DNA, 762 bp, Nostoc sp. PCC73102
atgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatga

SEQ ID NO: 73 - DNA, 617 bp, Haematococcus pluvialis
tagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgc

SEQ ID NO: 74 - DNA, 1208 bp, Haematococcus pluvialis
attgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgaattcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctag

SEQ ID NO: 75 - DNA, 6316 bp, Blakeslea trispora
  misc_feature (2694)..(2694): n is a, c, g, or t
aaggatgaagaatccaactctaataaaaatcttatggatatctttgatcgactcaaaaaggctttcaatgctattgctattaaaaaaaaagagagagagagaactatgagcaaaaggactctatgccaagatggcaaaaaggcaccagaaacccttagtttattattgcataatccagtcgagctagtacttctgtagctcaagcttaaccgaggatcttggaatcaactcgtctcgtcactcttgccgatgatcctagaaatggtatctatggatgttatactaacattgttatctttcaaggcctcgaagatgttattgttgcggtgataaataggctgctatgtactgaagttgctctgtaaaatgaatctagttcactgcctactcagcaaatggttgtttctaatgtctttaaagaaagaaaaaaagatacatatagactacccttcctttcaagactgtaatcgagaatcggccgatggtttattacaattagacgctgggaataagcaaaaggattcatctttgtaaataagagactggtgcatatgaaagcaaggatcgtatcaaggaatagttttgatcgagcatcaccagcaaatgctgctaatgttggcttcttctttgcttcctgagattgaatgggatgtgcctagagcattgctatttttaagtgtatactttagatttgtgtctttagatttgtgtcattttatttagtcaagaaagatccccctttctctatgtatgctaagaagaaggagcaagaagtgtatttacaagttggaatgagattgaaatattgtacataataataataaaaagaaaggtagatcaaaaaaaatgttctgcctattgtaagaaatcgggaccaacaggtgcttgataaccagaagtagcttccaattcaggtagaggctctagggacaaatacacaattatgacaggaattttcttgttgacttgaacactacaagagaaacgggtcagcacaaaatccgaaaaaaaaaagaaacggaccattcatgtcttacctatctagctctttgtcttcaattgcatcccattgctcaaccacagatacgcttcccaattgagtatattgatgaagtgttccctgcatttttcgcttgactaattccactacagtcacagtcttattaatgttttgtcctttaccagtcaggataatatgatctttttgcttcttctatcaaaaaaataattcttgttttgaataaaaaaaacaaatatttaaagaaactactttgatgacggtacctggaataactcgagacacacatctacatatgcgttgattttattgtggctaattcgaacctcattttctgctggtgggggctgttgactttcagttgctgagacgtccttcttgcttcttttatagtcttccactatgattttaatcaagaaagtaagtcagtgatgattgttacaagctatatatcttgaaaaagaacagagaggtattattatcagatgcaacatggttttctgtatcattttcatttcagtttctctgttcaaaaaaaaaaagaacactttctctttccactcctcaaattttttctgctaaactcctcgcaaaacatgtatttgctttaaactacaagttgcaattgtctgatttagcaatttcaatatgccttttgtgaatccacccaaaaataaacaagtgcttgagtatacttgggttcagttcaaaagaaagcaagcttttttttttctttcttgggaaagaaaaaaaaatattgttgagccatcctttaccagcagtatgcgagctacgacatagctggtctaacaatgactgcaagcaatagatcgagcttagtctttctattgcttcyttgtttgatctatgttcggccttacgctgacctatccaatactcgagataggcaacaagatttcgaacagtaatgaaataaatttcggataacagttgtggatgaggaagagaaagcgacttgaactcgagaaactttgttgaaatgaaatccgaccttttacgtgatcatcatgtattatcctctttttcttttttttcgtagtgaattacttactgattgcgctcaagtcgcgtctttataaagaagaaaaaaaaatattagaactttcaaaaaatataactgaaaataaaagtgtggctcggagagcaaataccacatcctttgtcttcgctttggtaacacggttaataagccactataggtgaataatgatcatttctgagaataaagcgcggcttgaagcttatatccatatcaggattcatattaggcacaactcacaattgaggttccagaagtgccaatttttttttcctgatagcctgtccaattaagatcaaaaaccactgagttttctctatatatttttttttttcataattcttaactcttcttcctctctctctctctctctctctttttggcttgcaaaaaaaatctttagtaataccaaagaaagcaaaccttttccttttcttatttccttgcttgttttttaatttttgatttctctatgctttaaatacccatttctttctttcttctgctattacctatcttttcattcctctcccccctctctctcttggtctataaacatcatgaagtcctcttttaaaagttcgcttgacatttatgctgtttatatacagcatcntgtgttttccaagtggttcattcttgcttttgttctttcgattttcctcaacacttatctactgaacgcttcgaagcaacagcccaaagtgataatcaaaaaggttattgagcgggtagaagtaccaagtagagaacaacctaaatcagtcataaagccctcctccaagaaacactcttctcatcatcagtctgatgtcattcgccctcttgatgaagtattgggtttgctcggaacacccgaggccttgactgatgaagagatcatctctattgttcaagctggtaaaatggccccctatgctcttgaaaaggtcttgggcgatttagagcgcgctgtccatatccgtcgtgctttgatctcccgtgactctcgtacgaaaactttggaagacagtatgcttcccgtgaaaaactatcattatgataaagtcatgggtgcttgttgtgaaaatgtcattggttatatgcctattccagtaggtgtcgcaggtaagaagttcaacaagtcgcgatatttgacaagttgctcatcattttcgaaacaggtcctttggtgattgatggtgattctattcatattcccatggcaactacggaaggttgtttagttgcttctactgccagaggttgtaaagcaatcaatgctggtggtggtgccaacacaattgttgttgctgatggtatgactcgaggtccttgtgtcgaatttcctacaatcactcgcgctgctgactgtaaacgatggattgaacaagagggtgaagctatcgtgaccgaggcattcaattcaacttctcgttttgctcgtgttcgtaaattgaaagttgctcttgccggtcgtctagtctacatccgtttctctaccactacaggtgatgcaatgggcatgaacatgatctccaagggttgtgaaaaggctttaagcaagattgctgagagatatcctgatatgcagatcatttctctttctggtaactattgtactgacaagaaacctgctgctatcaactggattgaaggacgtggtaaatctgttgttgctgasgctgtcatccctggtacggttgtcgaaaaggtattgaagacctctgttagtgctttggttgagctgaacatctctaaaaacctggttggttctgctatggctggctccgtcggtggctttaacgctcatgctgctaatattctaactgccatttaccttgctactggtcaagatcctgctcaaaatgtasagagttctaactgtattactttgatgaaagctgtcaatggcgaaagagaccttcatatctcttgtacaatgccctgtattgaagtaggcaccattggtggtggtactattttgcctcctcaacaagccatgttggatttcattggtgtgcgtggtcctcaccctaccgaacctggtgccaatgcccgwcgccttgctcgtgttatctgtgcctctgtgatggctggtgaattgtctttatgtgcagctttggctgctggtcatcttgtaaaggcacacatggctcataatcgtaataccactgctgctgccgctgttgttcctgcccctaanggcatagttgatgtctctacacctcctgctacacctgcagaaaagaatgatcctattcctggaagttgtatcaagtcatagaattaatattatatatatatcatatacaaaaaaaagaaaaaaaaaacactacatctatttatatttctccatgtacacacacacacacacatataaaaactctttattttccaatattttgcttttataaataatcttatttcattctaaataaactgtttttttttattaatcatcaaaccctgctgagagctgtgcaatatcatctatgttttcatggtttaactctggtatcggwcgagcctcctctgtacttgaagtttgtaggcagtttttatttaaggctgctggtcgatcatgatcatcakcaaacctgacagcatgaagttttgactgatgagcaatttcactaagggcagaatctgaactctttcgcttcctactattgaccatattgtctttaggtggaatgagtgaatagcgtcttgtcatatgtaacacagaatcaacaatatcctggtgatgaaactcggccaaacatagcgcctttctcccccaacaattataataatcaaaatgagaatgacatgtacggttttcctcgatgacaatatccaacgtcttgtcataatcctctgtgcgyataccattcatcttttggaagaacgcacggtagctctcacaagctgtcctcagagagttccgtgccatgtttcccaatgctcctggcaagtcgaaatgaagttgtcgaatctggcgatgtatgtctacaatgtcgcctgtttctttcattagatcaagcattcgtgtagcccaaatgatgtctatgttatgattttctttcattccagtaataactatagtttctcggcaaatcgaatgastgatggagtaaattcatcaaaagtgcaagtaatacatacagtgcttgaagaaatcttgtgtagcacgcctatattatgtaatataggatcgattctcgaaactcgacataaccaccaggctttagcaagcgttttatttcattcatgacaagctattgttaattcytgcttaataaaacaaaatgaaaaaaacatacccccctcmaaacttacttcccactcttgattggaaaaacaggtatagacgtgacgcatatgtatataatcaaaacactcatcaggatagggtaaaccattgagcacatcgcattgggtgaagaaagtattaggaggcttgatggctgtaggatatataggtgcaatatcaataccgtaaaactcagcatttgggaattctgtagccatctccagaatccaagtacctgtgccacaagcaacatcaagcactttaggtaagggtatacattgttgttcttgttgttgttgttgacaatcacttgagtctgagtttcgttttgattgttttaatgacaataattcttttacaggtgctgagaaattaccgtcaaatagatacttgtaaataaaatgctaaaaataaaaacaatagaaaaaaaaattgacgctcatttcattactatggaaataactgcaaaatcttaccacttgtacaagtctatcttgctcaatctcatcgtttggcagaatgtatttattgttgtagtattgatatcttctaccattcatgatataactgtcgcttctaatgctctgaggtgaagtacttgtaggtgaaggtggaagtgacgcaattttgtcaagcttaacaggatcctctcggctacatgttttctgcatatcaggaaaatcttgtttatttgaaacatcaacagtagatgtggtgtgatcttttttgaaaatatcgatgccttcctttgaaagccttttgaaaggctcttttaacttttttgagtgagagctacccatgatagcttatgaagaattaaaaagaaaaaagcaaaaaaaattaaaaaaaaaaaaagtagcaaaaaattctgtcgtaattatacaagccaatcaaaatcgaaattcatgcaaggcatagatgttcacgtggatttgatggttgatcctttttttttgcaaga

SEQ ID NO: 76 - DNA, 1170 bp, Thermus thermophilus
atgaagcgcctttccctgagggaggcctggccctacctgaaagacctccagcaagatcccctcgccgtcctgctggcgtggggccgggcccacccccggctcttccttcccctgccccgcttccccctggccctgatctttgaccccgagggggtggagggggcgctcctcgccgaggggaccaccaaggccaccttccagtaccgggccctctcccgcctcacggggaggggcctcctcaccgactggggggaaagctggaaggaggcgcgcaaggccctcaaagaccccttcctgccgaagaacgtccgcggctaccgggaggccatggaggaggaggcccgggccttcttcggggagtggcggggggaggagcgggacctggaccacgagatgctcgccctctccctgcgcctcctcgggcgggccctcttcgggaagcccctctccccaagcctcgcggagcacgcccttaaggccctggaccggatcatggcccagaccaggagccccctggccctcctggacctggccgccgaagcccgcttccggaaggaccggggggccctctaccgcgaggcggaagccctcatcgtccacccgcccctctcccaccttccccgagagcgcgccctgagcgaggccgtgaccctcctggtggcgggccacgagacggtggcgagcgccctcacctggtcctttctcctcctctcccaccgcccggactggcagaagcgggtggccgagagcgaggaggcggccctcgccgccttccaggaggccctgaggctctacccccccgcctggatcctcacccggaggctggaaaggcccctcctcctgggagaggaccggctccccccgggcaccaccctggtcctctccccctacgtgacccagaggctccacttccccgatggggaggccttccggcccgagcgcttcctggaggaaagggggaccccttcggggcgctacttcccctttggcctggggcagaggctctgcctggggcgggacttcgccctcctcgagggccccatcgtcctcagggccttcttccgccgcttccgcctagaccccctccccttcccccgggtcctcgcccaggtcaccctgaggcccgaaggcgggcttcccgcgcggcctagggaggaggtgcgggcgtga

SEQ ID NO: 77 - DNA, 2981 bp, Blakeslea trispora
tctagaattcattccattcgaaaggatcaacataaccaatttaatgactactagctaatggatacaaatatacgcacaaaaaaagaaagaattctatgatcaaagagaacacagacacagagtgatacatttaaatggttaagttcttatgatgttaaaatggtaactttattattgaattaaatgcgaatatcgttgctgctttgtacttggaaaacgttaggtaaaagttggttaatgaaagaagcaggagttgtagtatcatctcttgggaagaaatagaaaaagaggaaagtaacaaagtaacaagcaagacaataatagatccaatggctttcggtcttacgagtttgttcaggagcatacttcttttggctatcttgtaactttcttggtaagggattctggccaaagcttttacagacttggtcggaagtaagcttacttccagcaagaacgataggaacaccagtacctggatgtgtactacaaagaaaagagaaatgagtacgtgcgttattaaaaaaaagaaaaaaagagggcaaaagtattacctagctccgacaaagaaaagattatcataacggtttgtggaatccttggtactaggtctgaaccagagaacttggaacacatcatgagaaagaccaagaatagaacctctccaaaggttaaacttgctttgccaaacactaggatcattcacttcttcatgttcaatcaaattagcaaagttgtttactcccaaacgacgttcgataacttccagaaccatcttgcgtgcacggtttaccaactcaggataattttcttcagcactgtttcctgtcttactcttcatatggccaattggaaccaacacaataatggagtccttgttgggaggtgcggcagattcatcaattcgagatggaacgttgacatagaatgaagcttcagagggcaaaccgaagtcgttgaaaatctcatcaaaactttccttgtaggcttcagccaagaagatattgtgtacgtctaattgaggcacctttgttgacatggaccaataaaacgaaatagatgatgaagtgagtttctttgaggctaatgtcttctttgtccaattgcaaggaggtaacagatggtgataagcataaacaagatccgcattacatacgactgcatcggcttcaatgacttctccgctttccaaagtgacaccggttacacgcttgtctttatcgacagtgttaattttagcaacaggcgattgatatctgaattcagcaccgtactttttggaggcgatagactcaagcttctgaacaaccatgttgaaaccaccacgaggataccagataccttcagcaaactcggtgtattgtaacaaactgtaaactgctggagcatcataaggcgacatactatattccaaaaatagaaaatagaacaatgaatatcaaaattcctttcacttgccctttttcacatttctcttttcccacccccgaccggtctcactcatttttttttcatcccacaccacgcgttgtatgtgtacttaccccatatacattgtttgaaaagtaaaagccatacgcattttcttggtttggaaatatttactggctcggtcatagatcttaccaaacaagtgcaagcgaaagatttcaggcacatactgaagacgaatcaaatcccaaatggtttcaaagttgcgcttgatagcaataaatgtaccttgttcataatggacatgtgtttccttcatgaaatccaagaatctaccaaatccaaggggaccctcaatacggtccaattcgcccttcatcttggttaaatcggaagagagttgtacggcatcaccgtcgtcaaaatgaaccttatagttattgtcacagcgaagcaaatccaaatgatcaccaatacgttcatccaaatcagcaaatgcatcttcaaaaagcttaggcatcaaatagagtgagggaccctgatcaaagcgatgaccatcgtgatgaatgaatgaacaacggccaccggaaaagtcgttcttttcaacaacagtaactcgaaaaccttcacgagcaagacgagcagcagtagcagttccgccaataccggcaccaatgacaacaatatgcttcttttgatcagacatgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgtcaatactcacttatctggaatttcatctctactatacactacctgtccttgcggcattgtgttggctgctaaagccgtttcactcacagcaagacaatctcaagtataaatttttaatgttgatggccgcctctaccgcatcgatttgggacaattatatcgtttatcatcgcgcttggtggtactgtcctacttgtgttgtggctgtcattggctatgtacctctaga

SEQ ID NO: 78 - DNA, 1749 bp, Blakeslea trispora
atgtctgatcaaaagaagcatattgttgtcattggtgccggtattggcggaactgctactgctgctcgtcttgctcgtgaaggttttcgagttactgttgttgaaaagaacgacttttccggtggccgttgttcattcattcatcacgatggtcatcgctttgatcagggtccctcactctatttgatgcctaagctttttgaagatgcatttgctgatttggatgaacgtattggtgatcatttggatttgcttcgctgtgacaataactataaggttcattttgacgacggtgatgccgtacaactctcttccgatttaaccaagatgaagggcgaattggaccgtattgagggtccccttggatttggtagattcttggatttcatgaaggaaacacatgtccattatgaacaaggtacatttattgctatcaagcgcaactttgaaaccatttgggatttgattcgtcttcagtatgtgcctgaaatctttcgcttgcacttgtttggtaagatctatgaccgagccagtaaatatttccaaaccaagaaaatgcgtatggcttttacttttcaaacaatgtatatgggtatgtcgccttatgatgctccagcagtttacagtttgttacaatacaccgagtttgctgaaggtatctggtatcctcgtggtggtttcaacatggttgttcagaagcttgagtctatcgcctccaaaaagtacggtgctgaattcagatatcaatcgcctgttgctaaaattaacactgtcgataaagacaagcgtgtaaccggtgtcactttggaaagcggagaagtcattgaagccgatgcagtcgtatgtaatgcggatcttgtttatgcttatcaccatctgttacctccttgcaattggacaaagaagacattagcctcaaagaaactcacttcatcatctatttcgttttattggtccatgtcaacaaaggtgcctcaattagacgtacacaatatcttcttggctgaagcctacaaggaaagttttgatgagattttcaacgacttcggtttgccctctgaagcttcattctatgtcaacgttccatctcgaattgatgaatctgccgcacctcccaacaaggactccattattgtgttggttccaattggccatatgaagagtaagacaggaaacagtgctgaagaaaattatcctgagttggtaaaccgtgcacgcaagatggttctggaagttatcgaacgtcgtttgggagtaaacaactttgctaatttgattgaacatgaagaagtgaatgatcctagtgtttggcaaagcaagtttaacctttggagaggttctattcttggtctttctcatgatgtgttccaagttctctggttcagacctagtaccaaggattccacaaaccgttatgataatcttttctttgtcggagctagtacacatccaggtactggtgttcctatcgttcttgctggaagtaagcttacttccgaccaagtctgtaaaagctttggccagaatcccttaccaagaaagttacaagatagccaaaagaagtatgctcctgaacaaactcgtaagaccgaaagccattggatctattattgtcttgcttgttactttgttactttcctctttttctatttcttcccaagagatgatactacaactcctgcttctttcattaaccaacttttacctaacgttttccaagtacaaagcagcaacgatattcgcatttaa

SEQ ID NO: 79 - DNA, 25 bp, Artificial Sequence
  Primer
ccgatggcgacgacggaaggttgtt

SEQ ID NO: 80 - DNA, 25 bp, Artificial Sequence
  Primer
catgttcatgcccattgcatcacct

(19) United States
(12) Patent Application Publication
(10) Pub. No.: US 2006/0099670 A1
(43) Pub. Date: May 11, 2006

(54) Method for the genetic modification of organisms of the genus blakeslea, corresponding organisms and the use of the same

(76) Inventors: Markus Matuschek, Weinheim (DE); Thorsten Heinekamp, Hannover (DE); Andre Schmidt, Springe (DE); Axel Brakhage, Hannover (DE)

Correspondence Address: CONNOLLY BOVE LODGE & HUTZ, LLP, P O BOX 2207, WILMINGTON, DE, 19899, US

(21) Appl. No.: 10/541,993

(22) PCT Filed: Jan. 9, 2004
(86) PCT No.: PCT/EP04/00100

§ 371 Date: Jul. 8, 2005

(30) Foreign Application Priority Data
Jan. 9, 2003 (DE) 10300649.4
Sep. 8, 2003 (DE) 10341272.7

(51) Int. Cl. C12P 23/00; C12N 15/74; C12N 1/18

(52) U.S. Cl. 435/67; 435/483; 435/254.2

(57) ABSTRACT

The invention relates to a method for producing a genetically modified organism of the Blakeslea genus, which method comprises the following steps (i) transformation of at least one of the cells, (ii) optional homokaryotic conversion of the cells obtained in step (i) to produce cells, in which one or more genetic characteristics of the nuclei are all modified in an identical manner and said modification manifests itself in the cells, and (iii) selection and cultivation of the genetically modified cell or cells.

The invention relates to a method for the genetic modification of organisms of the Blakeslea genus, to corresponding organisms and to the use of the same.

Thus, for example, Blakeslea trispora is used as a producer organism for β-carotene (Ciegler, 1965, Adv Appl Microbiol. 7:1) and lycopene (EP 1201762, EP 1184464, WO 03/038064). In addition, Blakeslea is suitable for producing other lipophilic substances such as, for example, other carotenoids and their precursors, phospholipids, triacylglycerides, steroids, waxes, fat-soluble vitamins, provitamins and cofactors or for producing hydrophilic substances such as, for example, proteins, amino acids, nucleotides and water-soluble vitamins, provitamins and cofactors.

High productivities for β-carotene and lycopene render Blakeslea, in particular Blakeslea trispora, attractive for economic fermentative production of carotenoids and their precursors.

However, it is also of interest to further increase the productivities of carotenes and their precursors which have previously been produced naturally and to enable further carotenoids such as, for example, xanthophylls to be produced which have been produced by and isolated from Blakeslea only to a very low extent, if at all, previously.

Carotenoids are added to feedstuffs, foodstuffs, food supplements, cosmetics and medicaments. Carotenoids are used especially as pigments for coloring. Aside from this, the antioxidative action of carotenoids and other properties of these substances are utilized. The carotenoids are divided into the pure hydrocarbons, the carotenes and the oxygen-containing hydrocarbons, the xanthophylls. Xanthophylls such as canthaxanthin and astaxanthin are employed, for example, in the pigmentation of hens' eggs and fish (Britton et al. 1998, Carotinoids, Vol. 3, Biosynthesis and Metabolism). The carotenes β-carotene and lycopene are employed especially in human nutrition. β-Carotene, for example, is used as a colorant for beverages. Lycopene has disease-preventing action (Argwal and Rao, 2000, CMAJ 163:739-744; Rao and Argwal 1999, Nutrition Research 19:305-323). The colorless carotenoid precursor phytoene is especially suitable for applications as antioxidant.

Most of the carotenoids and their precursors which are employed as additives in the abovementioned applications are prepared by chemical synthesis. Said chemical synthesis is multistage, very complicated and causes high production costs. In contrast, fermentative processes are comparatively simple and based on inexpensive starting materials. Fermentative processes to produce carotenoids may be economically attractive and capable of competing with chemical synthesis, if the productivity of previous fermentative processes were increased or new carotenoids were able to be prepared on the basis of the known producer organisms.

A method for the genetic modification of Blakeslea trispora is required, in particular, if the intention is to utilize Blakeslea for producing xanthophylls, since these compounds are not synthesized naturally by Blakeslea.

Various DNA sequences of Blakeslea trispora are known already, in particular the DNA sequence coding for the genes of carotenoid biosynthesis from geranylgeranyl pyrophosphate to β-carotene (WO 03/027293).

Thus far, however, no methods for the genetically engineered modification of Blakeslea, in particular Blakeslea trispora, are known.

A method for the production of genetically modified fungi which has been successfully employed in some cases is Agrobacterium-mediated transformation. Thus, for example, the following organisms have been transformed by agrobacteria: Saccharomyces cerevisiae (Bundock et al., 1995, EMBO Journal, 14:3206-3214), Aspergillus awamori, Aspergillus nidulans, Aspergillus niger, Colletotrichum gloeosporioides, Fusarium solani pisi, Neurospora crassa, Trichoderma reesei, Pleurotus ostreatus, Fusarium graminearum (van der Toorren et al., 1997, EP 870835), Agraricus bisporus, Fusarium venenatum (de Groot et al., 1998, Nature Biotechnol. 16:839-842), Mycosphaerella graminicola (Zwiers et al. 2001, Curr. Genet. 39:388-393), Glarea lozoyensis (Zhang et al., 2003, Mol. Gen. Genomics 268:645-655), Mucor miehei (Monfort et al. 2003, FEMS Microbiology Lett. 244:101-106).

Of particular interest is a homologous recombination which involves as many sequence homologies as possible between the DNA to be introduced and the cellular DNA, so that it is possible to introduce or eliminate site-specifically genetic information in the genome of the recipient organism. Otherwise, the donor DNA will be integrated into the genome of the recipient organism by illegitimate or nonhomologous recombination which is not site-specific.

Agrobacterium-mediated transformation and subsequent homologous recombination of the transferred DNA have been detected previously for the following organisms: Aspergillus awamori (Gouka et al. 1999, Nature Biotech 17:598-601), Glarea lozoyensis (Zhang et al., 2003, Mol. Gen. Genomics 268:645-655), Mycosphaerella graminicola ((Zwiers et al. 2001, Curr. Genet. 39:388-393).

Another known method for transforming fungi is electroporation. Hill, Nucl. Acids. Res. 17:8011 has shown the integrative transformation of yeast by electroporation. Transformation of filamentous fungi has been described by Chakaborty and Kapoor (1990, Nucl. Acids. Res. 18:6737).

A “biolistic” method, i.e. the transfer of DNA by bombardment of cells with DNA-loaded particles, has been described, for example, for Trichoderma harzianum and Gliocladium virens (Lorito et al. 1993, Curr. Genet. 24:349-356).

However, it has not been possible previously to successfully employ these methods for specific genetic modification of Blakeslea and in particular Blakeslea trispora.

A particular difficulty in producing specifically genetically modified Blakeslea and Blakeslea trispora is the fact that their cells are multinuclear at all stages of the sexual and vegetative cell cycles. For example, spores of the Blakeslea trispora strains NRRL2456 and NRRL2457 were found to have an average of 4.5 nuclei per spore (Metha and Cerdá-Olmedo, 1995, Appl. Microbiol. Biotechnol. 42:836-838). As a consequence of this, the genetic modification is usually present only in one or a few nuclei, i.e. the cells are heterokaryotic.

If the genetically modified Blakeslea species, in particular Blakeslea trispora, are intended to be used for production, it is important, in particular in the case of gene deletion, that the genetic modification is present in all nuclei of the producer strains so as to make possible a stable and high synthetic performance without byproducts. The strains must consequently be homokaryotic with respect to said genetic modification.

A method of generating homokaryotic cells has been described only for Phycomyces blakesleeanus (Roncero et al., 1984, Mutat. Res. 125:195). According to the method described there, nuclei are eliminated in the cells by adding the mutagenic agent MNNG (N-methyl-N′-nitro-N-nitrosoguanidine) so as to obtain statistically a certain number of cells with only one functional nucleus. The cells are then subjected to a selection in which only mononuclear cells having a recessive selection marker can grow into a mycelium. The progeny of these selected cells are multinuclear and homokaryotic. An example of a recessive selection marker for Phycomyces blakesleanus is dar. dar⁺ strains absorb the toxic riboflavin analog 5-carbon-5-deazariboflavin, unlike dar⁻ strains (Delbrück et al. 1979, Genetics 92:27). Recessive mutants are selected by adding 5-carbon-5-deazariboflavin (DARF).

However, this method is unknown for Blakeslea, in particular Blakeslea trispora, and has in particular not been described in relation to a transformation.

It is an object of the present invention to provide a method which enables Blakeslea strains, in particular Blakeslea trispora, to be genetically modified. In addition, it is an object of the invention to provide a method which allows homokaryotic genetically modified strains to be produced. A further object of the invention is to provide cells which have been genetically modified accordingly.

This object is achieved by a method for producing a genetically modified organism of the Blakeslea genus, which method comprises the following steps:
(i) transformation of at least one of the cells,
(ii) optional homokaryotic conversion of the cells obtained in step (i) to produce cells in which one or more genetic characteristics of the nuclei are all modified in an identical manner and said genetic modification manifests itself in the cells, and
(iii) selection of the genetically modified cell or cells.

The method of the invention enables multinuclear cells of the Blakeslea fungi to be genetically modified in a specific and stable manner, in order to obtain in this way mycelium of cells with uniform nuclei. The cells are preferably those of fungi of the Blakeslea trispora species.

Transformation means the transfer of genetic information into the organism, in particular fungus. This should include any possible methods known to the skilled worker of introducing said information, in particular DNA, for example bombardment with DNA-loaded particles, transformation using protoplasts, microinjection of DNA, electroporation, conjugation or transformation of competent cells, chemicals or agrobacteria-mediated transformation. Genetic information means a gene section, a gene or a plurality of genes. The genetic information may be introduced into the cells, for example, with the aid of a vector or as free nucleic acid (e.g. DNA, RNA) and in any other manner, and either be incorporated into the host genome by recombination or be present in a free form in the cell. Particular preference is given here to homologous recombination.

The preferred transformation method is the transformation mediated by Agrobacterium tumefaciens. To this end, the donor DNA to be transferred is first inserted into a vector which (i) carries the T-DNA ends flanking the DNA to be transferred, (ii) includes a selection marker and (iii) has, if appropriate, promoters and terminators for gene expression of the donor DNA. Said vector is transferred into an Agrobacterium tumefaciens strain harboring a Ti plasmid containing the vir genes. vir genes are responsible for DNA transfer in Blakeslea. This two-vector system is used for transferring the DNA from Agrobacterium into Blakeslea. To this end, the Agrobacteria are first incubated in the presence of Acetosyringone. Acetosyringone induces the vir genes. Spores of Blakeslea trispora are then incubated together with the induced cells of Agrobacterium tumefaciens on Acetosyringone-containing medium and thereafter transferred to medium which enables selection of the transformants, i.e. of the genetically modified Blakeslea strains.

The term vector is used in the present application to refer to a DNA molecule which is used for introducing foreign DNA into and, if appropriate, propagating said foreign DNA in a cell (see also “vector” in Römpp Lexikon Chemie—CDROM Version 2.0, Stuttgart/New York: Georg Thieme Verlag 1999). In the present application, the term “vector” is intended to include plasmids, cosmids etc. which serve this purpose.

Expression means in the present application the transfer of genetic information, starting from DNA or RNA, to a gene product (here preferably carotenoids), and is also intended to include the term overexpression, meaning increased expression so as for a product which is already produced in the untransformed cell (wild type) to be increasingly produced or to form a large part of the entire cell content.

Genetic modification means the introduction of genetic information into a recipient organism so that said information is expressed in a stable manner and passed on during cell division. Homokaryotic conversion is then carried out, if appropriate, i.e. the production of cells which comprise only uniform nuclei, i.e. nuclei having the same genetic information content.

This homokaryotic conversion is in particular required if the genetic information introduced by transformation is recessive, i.e. does not manifest itself. However, if transformation results in the presence of dominant genetic information, i.e. if said information manifests itself, homokaryotic conversion is not absolutely necessary.

The homokaryotic conversion preferably comprises selecting the mononuclear spores. A small proportion of the Blakeslea trispora spores is by nature mononuclear so that these spores can be sorted out, if appropriate after specific labeling, for example staining, of the cell nuclei. This is preferably carried out using FACS (Fluorescence Activated Cell Sorting), on the basis of the lower fluorescence of the mononuclear cells.

Alternatively, the homokaryotic conversion can be carried out by first reducing the number of nuclei. To this end, a mutagenic agent may be employed, in particular N-methyl-N′-nitronitrosoguanidine (MNNG). High energy radiation such as UV radiation or X rays may also be used for reducing the number of nuclei. The subsequent selection may be carried out using the FACS method or recessive selection markers.

Selection means the selection of cells whose nuclei include the same genetic information, i.e. cells which have the same properties such as resistances or production or increased production of a product. Preference is given to using for selection, aside from the FACS method, 5-carbon-5-deazariboflavin (darf) and hygromycin (hyg) or 5′-fluororotate (FOA) and uracil.

The vector employed in the transformation (i) can be designed so as for the genetic information comprised in said vector to be integrated into the genome of at least one cell. In this connection, genetic information in the cell may be switched off.

The vector employed in the transformation (i) can, however, also be designed in such a way that the genetic information comprised in said vector is expressed in the cell, i.e. genetic information is introduced which is not present in the corresponding wild type or which is increased or overexpressed by said transformation.

The vector may comprise any genetic information for genetic modifications of organisms of the Blakeslea genus.

“Genetic information” means preferably nucleic acids whose introduction into the organism of the Blakeslea genus results in a genetic modification in organisms of the Blakeslea genus, i.e., for example, in causing, increasing or reducing enzyme activities in comparison with the starting organism.

The vector may comprise, for example, genetic information for producing lipophilic substances such as, for example, carotenoids and their precursors, phospholipids, triacylglycerides, steroids, waxes, fat-soluble vitamins, provitamins and cofactors or genetic information for producing hydrophilic substances such as, for example, proteins, amino acids, nucleotides and water-soluble vitamins, provitamins and cofactors.

The vector employed preferably comprises genetic information for producing carotenoids or xanthophylls or their precursors.

The vector preferably comprises genetic information causing the carotenoid biosynthesis enzymes to be located in the cell compartment in which carotenoid biosynthesis takes place.

Particular preference is given to genetic information for producing astaxanthin, zeaxanthin, echinenone, β-cryptoxanthin, andonixanthin, adonirubin, canthaxanthin, 3- and 3′-hydroxyechinenone, lycopene, lutein, β-carotene, phytoene or phytofluene. Very particular preference is given to genetic information for producing phytoene, bixin, lycopene, zeaxanthin, canthaxanthin and astaxanthin.

Accordingly, a preferred variant of the invention comprises producing and culturing organisms having an increased rate of synthesis of carotenoid biosynthesis intermediates and consequently increased productivity for final products of carotenoid biosynthesis. The rate of synthesis of carotenoid biosynthesis intermediates is increased in particular by increasing the activities of the enzymes 3-hydroxy-3-methylglutaryl coenzyme A reductase, isopentenyl pyrophosphate isomerase and geranyl pyrophosphate synthase.

Accordingly, a particularly preferred variant of the invention comprises producing and culturing organisms having an increased HMG-CoA reductase activity compared to the wild type.

HMG-CoA reductase activity means the enzyme activity of an HMG-CoA reductase (3-hydroxy-3-methylglutaryl coenzyme A reductase). HMG-CoA reductase means a protein which has the enzymic activity of converting 3-hydroxy-3-methylglutaryl coenzyme A to mevalonate.

Accordingly, HMG-CoA reductase activity means the amount of 3-hdroxy-3-methylglutaryl-coenzyme A converted or the amount of mevalonate produced by the protein HMG-CoA reductase within a particular time.

In the case of increased HMG-CoA reductase activity compared with the wild type, thus the protein HMG-CoA reductase increases the amount of 3-hydroxy-3-methylglutaryl coenzyme A converted or the amount of mevalonate produced within a particular time in comparison with the wild type.

This increase in HMG-CoA reductase activity is preferably at least 5%, more preferably at least 20%, more preferably at least 50%, more preferably at least 100%, more preferably at least 300%, still more preferably at least 500%, in particular at least 600%, of the HMG-COA reductase activity of the wild type.

In a preferred embodiment, the HMG-CoA reductase activity is increased compared to the wild type by increasing gene expression of a nucleic acid encoding an HMG-CoA reductase.

In a particularly preferred embodiment of the method of the invention, gene expression of a nucleic acid encoding an HMG-CoA reductase is increased by introducing into the organism a nucleic acid construct comprising a nucleic acid encoding an HMG-CoA reductase whose expression in said organism is subject to a reduced regulation, compared with the wild type.

Reduced regulation compared with the wild type means a reduced, preferably no, regulation at the expression or protein level in comparison with the wild type defined above.

Reduced regulation may preferably also be achieved by a promoter which is functionally linked to the coding sequence in the nucleic acid construct and which is subject to a reduced regulation in the organism, compared with the wild type promoter.

For example, the promoters ptef1 of Blakeslea trispora and pgpdA of Aspergillus nidulans are subject only to reduced regulation and are therefore particularly preferred promoters.

These promoters exhibit nearly constitutive expression in Blakeslea trispora so that transcriptional regulation no longer takes place via the intermediates of carotenoid biosynthesis.

In a further preferred embodiment, said reduced regulation can be achieved by using a nucleic acid encoding an HMG-CoA reductase, whose expression in said organism is subject to a reduced regulation, compared with the orthologous nucleic acid intrinsic to said organism.

Particular preference is given to using a nucleic acid which encodes only the catalytic region of HMG-CoA reductase (truncated (t-)HMG-CoA reductase). The membrane domain responsible for regulation is absent. The nucleic acid used is thus subject to reduced regulation and thus results in an increase of gene expression of HMG-CoA reductase.

In a particularly preferred embodiment, nucleic acids comprising the sequence SEQ ID. NO. 75 are introduced into Blakeslea trispora.

Further examples of HMG-CoA reductases and thus also of the t-HMG-CoA reductases reduced to the catalytic region or the encoding genes can readily be found, for example, from various organisms whose genomic sequence is known by homology comparisons of the sequences from databases with SEQ ID. NO. 75.

Further examples of HMG-CoA reductases and thus also of the t-HMG-CoA reductases reduced to the catalytic region or the encoding genes can furthermore readily be found, for example starting from the sequence SEQ ID. NO. 75, from various organisms whose genomic sequence is not known, by hybridization and PCR techniques in a manner known per se.

In a particularly preferred embodiment, said reduced regulation is achieved by using a nucleic acid encoding an HMG-CoA reductase, whose expression in said organism is subject to a reduced regulation, compared with the orthologous nucleic acid intrinsic to said organism, and using a promoter which is subject to a reduced regulation in said organism, compared with the wild type promoter.

Accordingly, a preferred variant of the invention comprises the transformation switching off phytoene desaturase gene expression, thus enabling the phytoene produced by the organisms to be isolated. The vector employed in the transformation (i) therefore comprises in one embodiment of the invention preferably a sequence coding for a fragment of the gene of phytoene desaturase, in particular Blakeslea trispora carB, with SEQ ID NO: 69.

Accordingly, a preferred variant of the invention comprises lycopene cyclase gene expression being switched off by transformation, thus enabling the lycopene produced by the organisms to be isolated. The vector employed in said transformation therefore comprises in one embodiment of the invention preferably a sequence coding for a fragment of the lycopene cyclase gene, in particular Blakeslea trisporas carR (WO 03/027293).

In a further preferred embodiment, the organisms of the Blakeslea genus are enabled, for example, to produce xanthophylls such as, for example, zeaxanthin or astaxanthin, by the genetically modified organisms of the Blakeslea genus having a hydroxylase activity and/or a ketolase activity, in comparison with the wild type.

Thus, in a further, preferred variant of the invention, the vector employed in the transformation (i) comprises genetic information which, after expression, displays a ketolase and/or hydroxylase activity so that the organisms produce zeaxanthin or astaxanthin.

Ketolase activity means the enzyme activity of a ketolase.

A ketolase means a protein which has the enzymic activity of introducing a keto group at the optionally substituted β-ionone ring of carotenoids.

A ketolase means in particular a protein which has the enzymic activity of converting β-carotene to canthaxanthin.

Accordingly, ketolase activity means the amount of β-carotene converted or the amount of canthaxanthin produced by the protein ketolase within a particular time.

According to the invention, the term “wild type” means the corresponding genetically unmodified starting organism of the Blakeslea genus.

The term “organism” may mean the starting organism (wild type) of the Blakeslea genus or a genetically modified organism according to the invention of the Blakeslea genus or both, depending on the context.

Preferably “wild type” for causing the ketolase activity and for causing the hydroxylase activity means in each case a reference organism.

This reference organism of the Blakeslea genus is Blakeslea trispora ATCC 14271 or ATCC 14272 which differ merely with respect to the mating type.

The ketolase activity in genetically modified organisms according to the invention of the Blakeslea genus and in wild type or reference organisms is preferably determined under the following conditions:

The ketolase activity in organisms of the Blakeslea genus is determined following the method of Fraser et al., (J. Biol. Chem. 272(10): 6128-6135, 1997). The ketolase activity in extracts is determined using the substrates beta-carotene and canthaxanthin in the presence of lipid (soya lecithin) and detergent (sodium cholate). Substrate-to-product ratios of the ketolase assays are determined by means of HPLC.

In this preferred embodiment, the genetically modified organism according to the invention of the Blakeslea genus has, in comparison with the genetically unmodified wild type, a ketolase activity and is thus preferably capable of transgenically expressing a ketolase.

In a further preferred embodiment, the ketolase activity in the organisms of the Blakeslea genus is caused by gene expression of a nucleic acid encoding a ketolase.

In this preferred embodiment, gene expression of a nucleic acid encoding a ketolase is preferably caused by introducing nucleic acids encoding ketolases into the starting organism of the Blakeslea genus.

For this purpose, it is possible in principle to use any ketolase gene, i.e. any nucleic acid encoding a ketolase.

Any of the nucleic acids mentioned in the description may be an RNA, DNA or cDNA sequence for example.

In the case of genomic ketolase sequences from eukaryotic sources, which include introns, preference is given to using already processed nucleic acid sequences such as the corresponding cDNAs, if the host organism of the Blakeslea genus is unable or cannot be made to express the corresponding ketolase.

Examples of nucleic acids encoding a ketolase and the corresponding ketolases, which may be used in the method of the invention, are, for example, sequences from:

Haematoccus pluvialis, in particular from Haematoccus pluvialis Flotow em. Wille (accession NO: X86782; nucleic acid: SEQ ID NO: 11, protein SEQ ID NO: 12),

Haematoccus pluvialis, NIES-144 (accession NO: D45881; nucleic acid: SEQ ID NO: 13, protein SEQ ID NO: 14),

Agrobacterium aurantiacum (accession NO: D58420; nucleic acid: SEQ ID NO: 15, protein SEQ ID NO: 16),

Alicaligenes spec. (accession NO: D58422; nucleic acid: SEQ ID NO: 17, protein SEQ ID NO: 18),

Paracoccus marcusii (accession NO: Y15112; nucleic acid: SEQ ID NO: 19, protein SEQ ID NO: 20),

Synechocystis sp. Strain PC6803 (accession NO: NP442491; nucleic acid: SEQ ID NO: 21, protein SEQ ID NO: 22),

Bradyrhizobium sp. (accession NO: AF218415; nucleic acid: SEQ ID NO: 23, protein SEQ ID NO: 24),

Nostoc sp. Strain PCC7120 (accession NO: AP003592, BAB74888; nucleic acid: SEQ ID NO: 25, protein SEQ ID NO: 26),

Nostoc punctiforme ATTC 29133, Nucleic acid: Acc. No. NZ_AABC01000195, base pair 55,604 to 55,392 (SEQ ID NO: 27); Protein: Acc. No. ZP_{—}00111258 (SEQ ID NO: 28) (annotated as putative protein) or

For example, the conditions during the washing step may be selected from the range of conditions limited by those of low stringency (with 2×SSC at 50° C.) and those of high stringency (with 0.2×SSC at 50° C., preferably at 65° C.) (20×SSC: 0.3 M sodium citrate, 3 M sodium chloride, pH 7.0).

An additional possibility is to rise the temperature during the washing step from moderate conditions at room temperature, 22° C., up to stringent conditions at 65° C.

Both parameters, the salt concentration and temperature, can be varied simultaneously, and it is also possible to keep one of the two parameters constant and vary only the other one. It is also possible to employ denaturing agents such as, for example, formamide or SDS during the hybridization. Hybridization in the presence of 50% formamide is preferably carried out at 42° C.

Some examples of conditions for hybridization and washing step are given below:

(1) hybridization conditions with, for example,

(i) 4×SSC at 65° C., or

(ii) 6×SSC at 45° C., or

(iii) 6×SSC at 68° C., 100 mg/ml denatured fish sperm DNA, or

(iv) 6×SSC, 0.5% SDS, 100 mg/ml denatured, fragmented salmon sperm DNA at 68° C., or

(v) 6×SSC, 0.5% SDS, 100 mg/ml denatured, fragmented salmon sperm DNA, 50% formamide at 42° C., or

(vi) 50% formamide, 4×SSC at 42° C., or

(vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or

(viii) 2× or 4×SSC at 50° C. (moderate conditions), or

(ix) 30 to 40% formamide, 2× or 4×SSC at 42° C. (moderate conditions).

(2) Washing steps of 10 minutes each with, for example,

(i) 0.015 M NaCl/0.0015 M sodium citrate/0.1% SDS at 50° C., or

(ii) 0.1×SSC at 65° C., or

(iii) 0.1×SSC, 0.5% SDS at 68° C., or

(iv) 0.1×SSC, 0.5% SDS, 50% formamide at 42° C., or

(v) 0.2×SSC, 0.1% SDS at 42° C., or

(vi) 2×SSC at 65° C. (moderate conditions).

In a preferred embodiment of the genetically modified organisms according to the invention of the Blakeslea genus, nucleic acids are introduced which encode a protein comprising the amino acid sequence SEQ ID NO: 12 or a sequence which is derived from this sequence by substitution, insertion or deletion of amino acids and which has an identity of at least 20%, preferentially at least 30%, preferably at least 40%, preferably at least 50%, preferably at least 60%, preferably at least 70%, preferably at least 80%, particularly preferably at least 90%, in particular 91%, 92%, 93%, 94%, 95%, 96%, 97%, 98%, 99%, at the amino acid level with the sequence SEQ ID NO: 12 and which has the enzymic property of a ketolase.

In this connection, it is possible for the ketolase sequence to be a natural one which can be found as described above by identity comparison of the sequences from other organisms, or for the ketolase sequence to be an artificial one which has been modified starting from the sequence SEQ ID NO: 12 by artificial variation, for example by substitution, insertion or deletion of amino acids.

A further, preferred embodiment of the methods of the invention involves introducing nucleic acids which encode a protein comprising the amino acid sequence SEQ ID NO: 26 or a sequence which is derived from this sequence by substitution, insertion or deletion of amino acids and which has an identity of at least 20%, preferentially at least 30%, preferably at least 40%, preferably at least 50%, preferably at least 60%, preferably at least 70%, preferably at least 80%, particularly preferably at least 90%, in particular 91%, 92%, 93%, 94%, 95%, 96%, 97%, 98%, 99%, at the amino acid level with the sequence SEQ ID NO: 26 and which has the enzymic property of a ketolase.

In this connection, it is possible for the ketolase sequence to be a natural one which can be found as described above by identity comparison of the sequences from other organisms, or for the ketolase sequence to be an artificial one which has been modified starting from the sequence SEQ ID NO: 26 by artificial variation, for example by substitution, insertion or deletion of amino acids.

A further, preferred embodiment of the methods of the invention involves introducing nucleic acids which encode a protein comprising the amino acid sequence SEQ ID NO: 30 or a sequence which is derived from this sequence by substitution, insertion or deletion of amino acids and which has an identity of at least 20%, preferentially at least 30%, preferably at least 40%, preferably at least 50%, preferably at least 60%, preferably at least 70%, more preferably at least 80%, particularly preferably at least 90%, in particular 91%, 92%, 93%, 94%, 95%, 96%, 97%, 98%, 99%, at the amino acid level with the sequence SEQ ID NO: 30 and which has the enzymic property of a ketolase.

In this connection, it is possible for the ketolase sequence to be a natural one which can be found as described above by identity comparison of the sequences from other organisms, or for the ketolase sequence to be an artificial one which has been modified starting from the sequence SEQ ID NO: 30 by artificial variation, for example by substitution, insertion or deletion of amino acids.

The term “substitution” means in the description substitution of one or more amino acids by one or more amino acids. Preference is given to carrying out “conservative” substitutions in which the replaced amino acid has a similar property to the original amino acid, for example substitution of Glu by Asp, Gln by Asn, Val by Ile, Leu by Ile, Ser by Thr.

Deletion is the replacement of an amino acid by a direct bond. Preferred positions for deletions are the termini of the polypeptide and the linkages between the individual protein domains.

Insertions are insertions of amino acids into the polypeptide chain, with formal replacement of a direct bond by one or more amino acids.

Identity between two proteins means the identity of the amino acids over the entire length of each protein, in particular the identity calculated by comparison with the aid of Lasergene software from DNASTAR, inc. Madison, Wis. (USA) using the Clustal method (Higgins D G, Sharp P M. Fast and sensitive multiple sequence alignments on a microcomputer. Comput Appl. Biosci. 1989 April;5(2):151-1), setting the following parameters:

Multiple alignment parameter:

| | |
|---|---|
| Gap penalty | 10 |
| Gap length penalty | 10 |

Pairwise alignment parameter:

| | |
|---|---|
| K-tuple | 1 |
| Gap penalty | 3 |
| Window | 5 |
| Diagonals saved | 5 |

Accordingly, a protein which has an identity of at least 20% at the amino acid level with the sequence SEQ ID NO: 12 or 26 or 30 means a protein which, on comparison of its sequence with the sequence SEQ ID NO: 12 or 26 or 30, in particular using the above program logarithm with the above set of parameters, has an identity of at least 20%, preferably 80%, 85%, particularly 90%, in particular 95%.

Suitable nucleic acid sequences can be obtained, for example, by back translation of the polypeptide sequence in accordance with the genetic code.

The codons preferably used for this purpose are those frequently used according to the Blakeslea-specific codon usage. The codon usage can easily be found by means of computer analyses of other, known genes of organisms of the Blakeslea genus.

In a particularly preferred embodiment, a nucleic acid comprising the sequence SEQ ID NO: 11 is introduced into the organism of said genus.

In a particularly preferred embodiment, a nucleic acid comprising the sequence SEQ ID NO: 25 is introduced into the organism of said genus.

In a particularly preferred embodiment, a nucleic acid comprising the sequence SEQ ID NO: 29 is introduced into the organism of said genus.

All the aforementioned ketolase genes can moreover be prepared in a manner known per se by chemical synthesis from the nucleotide building blocks, for example by fragment condensation of individual overlapping, complementary nucleic acid building blocks of the double helix. Chemical synthesis of oligonucleotides is possible, for example, in a known manner by the phosphoamidite method (Voet, Voet, 2nd edition, Wiley Press New York, pages 896-897). Addition of synthetic oligonucleotides and filling in of gaps with the aid of the Klenow fragment of DNA polymerase and ligation reactions, and also general cloning methods are described in Sambrook et al. (1989), Molecular cloning: A laboratory manual, Cold Spring Harbor Laboratory Press.

The vector employed in the transformation (i) therefore comprises in one embodiment of the invention preferably a sequence coding for a ketolase, in particular the Nostoc punctiforme ketolase with SEQ ID NO: 72.

Hydroxylase activity means the enzymic activity of a hydroxylase.

A hydroxylase means a protein having the enzymic activity of introducing a hydroxyl group on the, optionally substituted, β-ionone ring of carotenoids.

In particular, a hydroxylase means a protein having the enzymic activity of converting β-carotene to zeaxanthin or cantaxanthin to astaxanthin.

Accordingly, hydroxylase activity means the amount of β-carotene or cantaxanthin converted, or amount of zeaxanthin or astaxanthin produced, by the hydroxylase protein in a particular time.

Thus, when the hydroxylase activity is increased compared with the wild type, the amount of β-carotene or canthaxantin converted or the amount of zeaxanthin or astaxanthin produced in a particular time by the hydroxylase protein is increased in comparison with the wild type.

This increase in hydroxylase activity is preferably at least 5%, further preferably at least 20%, further preferably at least 50%, further preferably at least 100%, more preferably at least 300%, still more preferably at least 500%, in particular at least 600%, of the hydroxylase activity of the wild type.

The hydroxylase activity in the genetically modified organisms of the invention and in wild-type and reference organisms is preferably determined under the following conditions:

The hydroxylase activity is determined by the method of Bouvier et al. (Biochim. Biophys. Acta 1391 (1998), 320-328) in vitro. Ferredoxin, Ferredoxin-NADP oxidoreductase, katalase, NADPH and beta-carotene are added with mono- and digalactosyl glycerides to a defined amount of organism extract.

The hydroxylase activity is particularly preferably determined under the following conditions of Bouvier, Keller, d'Harlingue and Camara (Xanthophyll biosynthesis: molecular and functional characterization of carotenoid hydroxylases from pepper fruits (Capsicum annuum L.; Biochim. Biophys. Acta 1391 (1998), 320-328):

The in vitro assay is carried out in a volume of 0.250 ml. The mixture contains 50 mM potassium phosphate (pH 7.6), 0.025 mg of spinach ferredoxin, 0.5 unit of spinach ferredoxin-NADP+ oxidoreductase, 0.25 mM NADPH, 0.010 mg of beta-carotene (emulsified in 0.1 mg of Tween 80), 0.05 mM of a mixture of mono- and digalactosyl glycerides (1:1), 1 unit of catalysis, 200 mono- and digalactosyl glycerides, (1:1), 0.2 mg of bovine serum albumin and organism extract in a varying volume. The reaction mixture is incubated at 30° C. for 2 hours. The reaction products are extracted with an organic solvent such as THF, acetone or chloroform/methanol (2:1) and determined by HPLC.

The hydroxylase activity is particularly preferably determined under the following conditions of Bouvier, d'Harlingue and Camara (Molecular Analysis of carotenoid cyclae inhibition; Arch. Biochem. Biophys. 346(1) (1997) 53-64):

The in vitro assay is carried out in a volume of 250 μl. The mixture contains 50 mM potassium phosphate (pH 7.6), varying amounts of organism extract, 20 nM lycopene, 250 μg of paprika chromoplastid stromal protein, 0.2 mM NADP+, 0.2 mM NADPH and 1 mM ATP. NADP/NADPH and ATP are dissolved in 10 ml of ethanol with 1 mg of Tween 80 immediately before addition to the incubation medium. After a reaction time of 60 minutes at 30° C., the reaction is stopped by adding chloroform/methanol (2:1). The reaction products extracted into chloroform are analyzed by HPLC.

An alternative assay with radioactive substrate is described in Fraser and Sandmann (Biochem. Biophys. Res. Comm. 185(1) (1992) 9-15).

The hydroxylase activity can be increased in various ways, for example by switching off inhibitory regulatory mechanisms at the expression and protein levels or by increasing gene expression of nucleic acids encoding a hydroxylase, compared with the wild type.

Gene expression of the nucleic acids encoding a hydroxylase can likewise be increased, compared with the wild type, in various ways, for example by inducing the hydroxylase gene by activators or by introducing one or more hydroxylase gene copies, i.e. by introducing at least one nucleic acid encoding a hydroxylase into the organism of the Blakeslea genus.

In a preferred embodiment, gene expression of a nucleic acid encoding a hydroxylase is increased by introducing at least one nucleic acid encoding a hydroxylase into the organism of the Blakeslea genus.

It is possible to use for this purpose in principle any hydroxylase gene, i.e. any nucleic acid which encodes a hydroxylase.

In the case of genomic hydroxylase sequences from eukaryotic sources, which comprise introns, preference is given to using nucleic acid sequences which have already been processed, such as the corresponding cDNAs, if the host organism is unable or cannot be made to express the corresponding hydroxylase.

One example of a hydroxylase gene is a nucleic acid encoding a Haematococcus pluvialis hydroxylase, with accession No. AX038729 (WO 0061764; nucleic acid: SEQ ID NO: 31, protein: SEQ ID NO: 32), an Erwinia uredovora 20D3 hydroxylase (ATCC 19321, accession No. D90087; nucleic acid: SEQ ID NO: 33, protein: SEQ ID NO: 34) or Thermus thermophilus hydroxylase (DE 102 34 126.5) encoded by the sequence SEQ ID NO 76.

Further hydroxylases are encoded by the nucleic acids having the following accession numbers

|emb|CAB55626.1, CAA70427.1, CAA70888.1, CAB55625.1,
AF499108_1, AF315289_1, AF296158_1, AAC49443.1,
NP_194300.1, NP_200070.1, AAG10430.1, CAC06712.1,
AAM88619.1, CAC95130.1, AAL80006.1, AF162276_1,
AAO53295.1, AAN85601.1, CRTZ_ERWHE, CRTZ_PANAN,
BAB79605.1, CRTZ_ALCSP, CRTZ_AGRAU, CAB56060.1,
ZP_00094836.1, AAC44852.1, BAC77670.1, NP_745389.1,
NP_344225.1, NP_849490.1, ZP_00087019.1, NP_503072.1,
NP_852012.1, NP_115929.1, ZP_00013255.1

Thus, in this preferred embodiment, at least one hydroxylase gene is present in the preferred transgenic organisms according to the invention of the Blakeslea genus, compared with the wild type.

In this preferred embodiment, the genetically modified organism has, for example, at least one exogenous nucleic acid encoding a hydroxylase.

In the preferred embodiment described above, preference is given to using as hydroxylase genes nucleic acids which encode proteins comprising the amino acid sequence SEQ ID NO: 32, 34 or encoded by the sequence SEQ ID NO 76 or a sequence which is derived from this sequence by substitution, insertion or deletion of amino acids and which has an identity of least 30%, preferably at least 50%, more preferably at least 70%, still more preferably at least 80%, more preferably at least 90%, in particular 91%, 92%, 93%, 94%, 95%, 96%, 97%, 98%, 99%, at the amino acid level to the sequence SEQ. ID. NO: 32, 34, or encoded by the sequence with SEQ ID NO 76, and which have the enzymic property of a hydroxylase.

Further examples of hydroxylases and hydroxylase genes can readily be found, for example, from various organisms whose genomic sequence is known, as described above, by homology comparisons of the amino acid sequences or of the corresponding back-translated nucleic acid sequences from databases with SEQ ID. NO: 31, 33 or 76.

Further examples of hydroxylases and hydroxylase genes can furthermore readily be found in a manner known per se, for example starting from the sequence SEQ ID NO: 31, 33 or 76, from various organisms whose genomic sequence is unknown, as described above, by hybridization and PCR techniques.

In a further particularly preferred embodiment, nucleic acids which encode proteins comprising the amino acid sequence of the hydroxylase of sequence SEQ ID NO: 32, 34 or encoded by the sequence SEQ ID NO 76 are introduced into organisms to increase the hydroxylase activity.

Suitable nucleic acid sequences can be obtained, for example, by back translation of the polypeptide sequence in accordance with the genetic code.

Preference is given to using for this purpose those codons which are frequently used in accordance with the organism-specific codon usage. The codon usage can readily be determined on the basis of computer analyses of other, known genes of the organisms in question.

In a particularly preferred embodiment, a nucleic acid comprising the sequence SEQ. ID. NO: 31, 33 or 76 is introduced into the organism.

All the aforementioned hydroxylase genes can furthermore be prepared in a manner known per se by chemical synthesis from the nucleotide building blocks, for example by fragment condensation of individual overlapping, complementary nucleic acid building blocks of the double helix. Chemical synthesis of oligonucleotides is possible, for example, in a known manner by the phosphoamidite method (Voet, 2nd edition, Wiley Press New York, pages 896-897). Addition of synthetic oligonucleotides and filling in of gaps with the aid of the Klenow fragment of DNA polymerase and ligation reactions, and also general cloning methods are described in Sambrook et al. (1989), Molecular cloning: A laboratory manual, Cold Spring Harbor Laboratory Press.

The vector employed in the transformation (i) therefore comprises in a further embodiment of the invention preferably a sequence coding for a hydroxylase, in particular a Haematococcus pluvialis hydroxylase with SEQ ID NO: 70 or an Erwinia uredova hydroxylase with SEQ ID NO: 71 or a Thermus thermophilus hydroxylase encoded by the sequence SEQ ID NO 76.

The vector employed in the transformation (i) preferably also includes regions which control and support expression, in particular promoters and terminators.

The vector employed in the transformation (i) preferably includes the gpd and/or the ptef1 promoter and/or the trpC terminator, all of which have proved to be particularly successful in the transformation of Blakeslea. The use of “inverted repeats” familiar to the skilled worker (IR, Römpp Lexikon der Biotechnologie 1992, Thieme Verlag Stuttgart, page 407 “Inverse repetitive sequences”) for controlling expression and transcription is also within the scope of the invention.

The gpd promoter employed in the vector has advantageously the sequence SEQ ID NO: 1. The trpC terminator employed in the vector has advantageously the sequence SEQ ID NO: 2. The ptef1 promoter employed in the vector has advantageously the sequence SEQ ID NO: 35.

Preference is given here to using in particular the gpd promoter and the trpC terminator from Aspergillus nidulans and the ptef1 promoter from Blakeslea trispora.

The vector employed in the transformation (i) in particular comprises a resistance gene. The latter is preferably a hygromycin resistance gene (hph), in particular that from E. coli. This resistance gene has proved particularly suitable in the detection of transformation and selection of the cells.

The preferred promoter utilized for hph thus is p-gpdA, the promoter of glyceraldehyde 3-phosphate dehydrogenase coding for Aspergillus nidulans. The preferred terminator utilized for hph is t-trpC, the terminator of the trpC gene coding for Aspergillus nidulans anthranilate synthase components.

Derivatives of the pBinAHyg vector have proved to be particularly suitable vectors. The vector employed for transformation thus preferably comprises SEQ ID NO: 3. To this will be added, depending on the desired carotenoid or its precursor, a sequence coding for a hydroxylase, ketolase, phytoene desaturase etc., as described above. The vectors thus comprise in one embodiment of the invention the sequence SEQ ID NO: 69 coding for said phytoene desaturase. The vectors also comprise in a further embodiment of the invention the sequence SEQ ID NO: 72 coding for a ketolase. The vectors further comprise in a further embodiment of the invention the sequence SEQ ID NO: 70 or 71 or 76 coding for a hydroxylase. Corresponding combinations of the abovementioned sequences are also within the scope of the invention. Thus, the vector comprises in one embodiment both a sequence SEQ ID NO: 72 coding for a ketolase and the sequence SEQ ID NO: 70 or 71 or 76 coding for a hydoxylase and thus enables astaxanthin to be produced.

In particular, it is possible to use within the scope of the invention vectors selected from the group consisting of SEQ ID NO: 37 to 51 and 62.

The method of the invention enables genetically modified Blakeslea organisms, in particular of the Blakeslea trispora species, or mycelium formed by them to be obtained.

The genetically modified organisms may be used for producing carotenoids, xanthophylls or their precursors, in particular phytoene, bixin, astaxanthin, zeaxanthin and canthaxanthin. It is also possible, by introducing the appropriate genetic information, for new carotenoids which do not occur naturally in the wild type to be generated by the specifically genetically modified cells or by the mycelium formed thereby and subsequently to be isolated.

Preference is given to obtaining carotenoids or their precursors using the specifically genetically modified cells or the mycelium formed thereby.

If the genetic modification is carried out only in cells of one of the mating types found ((+) or (−) for Blakeslea trispora), the corresponding other, unmodified mating type is added to the cultivation, since it is possible in this way to achieve good production of the carotenoids or their precursors, owing to the substances released by the second, unmodified mating type (e.g. trisporic acids). Advantageously, however, the genetic modification is carried out in cells of both mating types which are then cultured together, thereby achieving particularly good growth and optimal production of the carotenoids or their precursors. An (artificial) addition of trisporic acids is possible and useful.

Trisporic acids are sex hormones in Mucorales fungi such as Blakeslea, which stimulate the formation of zygophores and production of β-carotene (van den Ende 1968, J. Bacteriol. 96:1298-1303, Austin et al. 1969, Nature 223:1178-1179, Reschke Tetrahedron Lett. 29:3435-3439, van den Ende 1970, J. Bacteriol. 101:423-428).

Materials and Methods

Molecular genetics work was carried out, unless described otherwise, by the methods in Current Protocols in Molecular Biology (Ausubel et al., 1999, John Wiley & Sons).

Strains and Growth Conditions

The Blakeslea trispora strains ATCC 14271 (mating type (+)) and ATCC14272 (−) mating type (−)) were obtained from the American Type Culture Collection. B. trispora were grown in MEP medium (malt extract-peptone medium): 30 g/l malt extract (Difco), 3 g/l peptone (Soytone, Difco), 20 g/l agar, pH set to 5.5, ad 1000 ml with H₂O at 28° C.

Agrobacterium tumefaciens LBA4404 were grown according to Hoekema et al. (1983, Nature 303:179-180) at 28° C. for 24 h in agrobacterial minimal medium (AMM) : 10 mM K₂HPO₄, 10 mM KH₂PO₄, 10 MM glucose, MM salts (2.5 mM NaCl, 2 mM MgSO₄, 700 μM CaCl₂, 9 μM FeSO₄, 4 mM (NH₄)₂SO₄).

Transformation of Agrobacterium tumefaciens

The plasmid pBinAHyg was electroporated into the agrobacterial strain LBA 4404 (Hoekema et al., 1983, Nature 303:179-180) (Mozo and Hooykaas, 1991, Plant Mol. Biol. 16:917-918). The following antibiotics were used for selection during agrobacterial growth: Rifampicin 50 mg/l (selection for the A. tumefaciens chromosome), streptomycin 30 mg/l (selection for the helper plasmid) and kanamycin 100 mg/l (selection for the binary vector).

Transformation of Blakeslea trispora

After 24 h of growth in AMM, the agrobacteria were diluted for transformation to an OD₆₀₀ of 0.15 in induction medium (IM: MM salts, 40 mM MES (pH 5.6), 5 mM glucose, 2 mM phosphate, 0.5% glycerol, 200 μM acetosyringone) and grown again in IM to an OD₆₀₀ Of approx. 0.6 overnight.

For coincubation of Blakeslea ATCC 14271 or ATCC14272 and Agrobacterium, 100 μl of agrobacterial suspension were mixed with 100 μl of Blakeslea spore suspension (10⁷ spores/ml in 0.9% NaCl) and distributed in a sterile manner on a nylon membrane (Hybond N, Amersham) on IM-agarose plates (IM+18 g/l agar). After 3 days of incubation at 26° C., the membrane was transferred to an MEP-agar plate (30 g/l malt extract, 3 g/l peptone, pH 5.5, 18 g/l agar). To select for transformed Blakeslea cells, the medium comprised hygromycin at a concentration of 100 mg/l and, to select against agrobacteria, 100 mg/l cefotaxime. The incubation was carried out at 26° C. for approx. 7 days. This was followed by transferring mycelium to fresh selection plates. Resultant spores were rinsed with 0.9% NaCl and plated on CM17-1 agar (3 g/l glucose, 200 mg/l L-asparagine, 50 mg/l MgSO₄×7H₂O, 150 mg/l KH₂PO₄, 25 μg/l thiamine-HCl, 100 mg/l Yeast Extract, 100 mg/l sodium deoxycholate, 100 mg/L hygromycin, 100 mg/L cefotaxime, pH 5.5, 18 g/l agar). Individual genetically modified spores were isolated by putting them individually on selection medium, using an FACS instrument from BectonDickson (Modell Vantage+Diva Option).

Preparation of Genetically Modified Blakeslea trispora by Agrobacterium-Mediated Transformation Preparation of the Recombinant Plasmid pBinAHyg

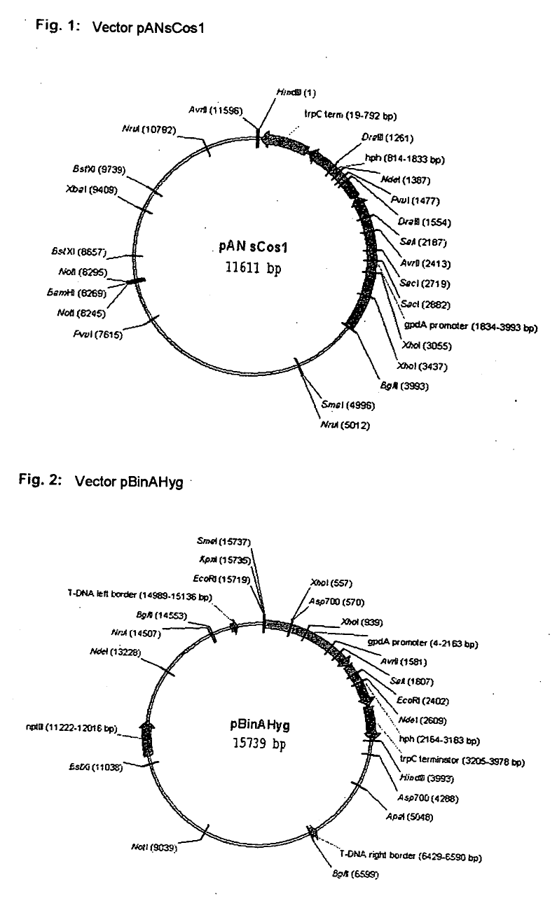

The gpdA-hph-trpC-cassette was isolated as BglII/HindIII fragment from the plasmid pANsCos1 (FIG. 1, Osiewacz, 1994, Curr. Genet. 26:87-90, SEQ ID NO: 4) and ligated into the binary plasmid pBin19 (Bevan, 1984, Nucleic Acids Res. 12:8711-8721) opened with BamHI/HindIII. The vector obtained in this way was referred to as pBinAHyg (FIG. 2, SEQ ID NO: 3) and comprised the E. coli hygromycin resistance gene (hph) under the control of the gpd promoter (SEQ ID NO: 1) and the trpC terminator (SEQ ID NO: 2) from Aspergillus nidulans and the corresponding border sequences required for Agrobacterium DNA transfer. The vectors mentioned in the exemplary embodiments described hereinbelow are pBinAHyg derivatives.

Transfer of pBinAHyg and pBinAHyg Derivatives into Agrobacterium tumefaciens

The transfer of the pBinAHyg plasmid into agrobacteria is described by way of example below. The derivatives were transferred in a similar manner.

The plasmid pBinAHyg was electroporated into the agrobacterial strain LBA 4404 (Hoekema et al., 1983, Nature 303:179-180) (Mozo and Hooykaas, 1991, Plant Mol. Biol. 16:917-918). The following antibiotics were used for selection during agrobacterial growth: Rifampicin 50 mg/l (selection for the A. tumefaciens chromosome), streptomycin 30 mg/l (selection for the helper plasmid) and kanamycin 100 mg/l (selection for the binary vector).

Transfer of pBinAHyg and pBinAHyg Derivatives into Blakeslea trispora

After 24 h of growth in AMM, the agrobacteria were diluted for transformation to an OD₆₆₀ of 0.15 in induction medium (IM: MM salts, 40 mM MES (pH 5.6), 5 mM glucose, 2 mM phosphate, 0.5% glycerol, 200 μM acetosyringone) and grown again in IM to an OD₆₆₀ of approx. 0.6 overnight.

For coincubation of Blakeslea trispora (B.t.) and Agrobacterium tumefasciens (A.t.) 100 μl of agrobacterial suspension were mixed with 100 μl of Blakeslea spore suspension (10⁷ spores/ml in 0.9% NaCl) and distributed in a sterile manner on a nylon membrane (Hybond N, Amersham) on IM-agarose plates (IM+18 g/l agar). After 3 days of incubation at 26° C., the membrane was transferred to an MEP-agar plate (30 g/l malt extract, 3 g/l peptone, pH 5.5, 18 g/l agar).

To select for transformed Blakeslea cells, the medium contained hygromycin at a concentration of 100 mg/l and, to select against agrobacteria, 100 mg/l cefotaxime. The incubation was carried out at 26° C. for approx. 7 days. This was followed by transferring mycelium to fresh selection plates. Resultant spores were rinsed with 0.9% NaCl and plated on CM17-1 agar (3 g/l glucose, 200 mg/l L-asparagine, 50 mg/l MgSO₄×7H₂O, 150 mg/l KH₂PO₄, 25 μg/l thiamine-HCl, 100 mg/l Yeast Extract, 100 mg/l sodium deoxycholate, pH 5.5, 100 mg/L cefotaxime, 100 mg/L hygromycine, 18 g/l agar). The transfer of spores to fresh selection plates was repeated three times. In this way, the transformant Blakeslea trispora GMO 3005 was isolated. Alternatively, the GMO (genetically modified organisms) were selected by applying the spores individually to CM-17 agar containing 100 mg/l cefotaxime, 100 mg/l hygromycin, by means of the BectonDickinson FacsVantage+Diva Option. In this case, fungal mycelium formed only where the spores had been genetically modified.

Detection of the Genetic Modification Due to Transfer of pBinAHyg and pBinAHyg Derivatives in Blakeslea trispora

Detection of the transfer is described by way of example below for pBinAHyg in Blakeslea trispora. Detection of the transfer of the derivatives was carried out in a similar manner.

200 ml of MEP medium (30 g/l malt extract, 3 g/l peptone, pH 5.5) were inoculated with 10⁵ to 10⁷ spores of the Blakeslea trispora GMO 3005 transformant and incubated on a rotary shaker at 200 rpm and 26° C. for 7 days. To detect successful transformation, DNA was isolated from the mycelium (Peqlab Fungal DNA Mini Kit) and used in a PCR (program: 94° C. for 1 min, then 30 cycles of 1 min. at 94° C., 1 min. at 58° C., 1 min. at 72° C., each).

The primers hph-forward (5′-CGATGTAGGAGGGCGTGGATA, SEQ ID NO: 5) and hph-reverse (5′-GCTTCTGCGGGCGATTTGTGT, SEQ ID NO: 6) were used for detecting the hygromycin resistance gene (hph). The expected hph fragment was 800 bp in length.

The primers nptIII-forward (5′-TGAGAATATCACCGGAATTG, SEQ ID NO: 7) and nptIII-reverse (5′-AGCTCGACATACTGTTCTTCC, SEQ ID NO: 8) were used for amplification of the kanamycin resistance gene nptIII and thus as a control for agrobacteria. The expected nptIII fragment was 700 bp in length.

The primers MAT292 (5′-GTGAATGGAAATCCCATCGCTGTC, SEQ ID NO: 9) and MAT293 (5′-AGTGGGTACTCTAAAGGCCATACC, SEQ ID NO: 10) were used for amplification of a fragment of the glycerinaldehyde 3-phosphate dehydrogenase gene gpd1 and thus as a control for Blakeslea trispora. The expected gpd1 fragment was 500 bp in length.

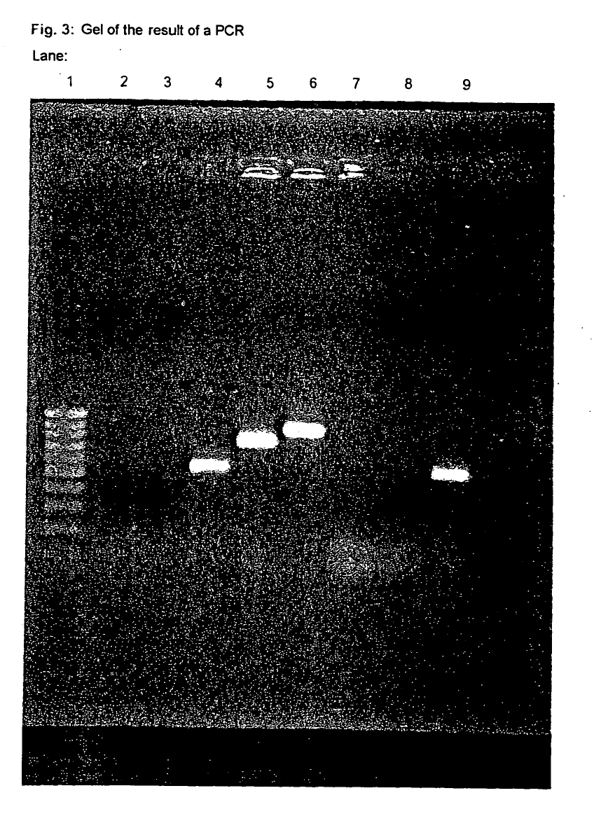

FIG. 3 depicts the result of the PCR of Blakeslea trispora DNA on the basis of a standard gel. The gel lanes were loaded as follows:

| 1) | 100 bp size marker (100 bp - 1 kb) | |
|---|---|---|
| 2) | B.t. GMO 3005 | primer nptIII-for / nptIII-rev |
| 3) | B.t. GMO 3005 | primer hph-for / hph-rev |
| 4) | B.t. GMO 3005 | primer MAT292 / MAT293 (gpd) |
| 5) | A.t. with pBinAHyg | primer nptIII-for / nptIII-rev |
| | plasmid | |
| 6) | A.t. with pBinAHyg | primer hph-for / hph-rev |
| | plasmid | |
| 7) | B.t. 14272 WT | primer nptIII-for / nptIII-rev |
| 8) | B.t. 14272 WT | primer hph-for / hph-rev |
| 9) | B.t. 14272 WT | primer MAT292 / MAT293 (gpd) |

The hygromycin resistance gene (hph) and, as a positive control, the glycerinaldehyde 3-phosphate dehydrogenase gene (gpd1) were detected in Blakeslea trispora DNA. In contrast, nptIII was not detected.

Thus, the genetic modification of Blakeslea trispora by Agrobacterium-mediated transformation was detected.

Isolation of homokaryotic Blakeslea trispora GMOs: The successful transfer of the pBinAHyg vector and pBinAHyg derivatives into Blakeslea trispora produces genetically modified organisms (GMO) of Blakeslea trispora. However, Blakeslea has multinuclear cells at all stages of the vegetative and sexual cell cycle. Therefore, foreign DNA is usually inserted only in one nucleus. It is the aim to obtain Blakeslea strains in which foreign DNA has been inserted in all nuclei, i.e. the aim is a homonuclear recombinant fungal mycelium.

1) Preparation of Homonuclear Recombinant Strains by Means of FACS (Fluorescence-Activated Cell Sorting)

A small proportion of the spores of Blakeslea trispora or of the genetically modified Blakeslea trispora strains is by nature mononuclear. To produce homonuclear recombinant strains comprising the foreign DNA of pBinAHyg or pBinAHyg derivatives, the mononuclear spores were sorted out by means of FACS and plated on MEP (30 g/l malt extract, 3 g/l peptone, pH 5.5, 18 g/l agar) containing 100 mg/l cefotaxime and 100 mg/l hygromycin. The mycelia produced here were homonuclear. For FACS, the spores of a 3 day old smear were washed off with 10 ml of Tris-HCl 50 mMol+0.1% Span20 per agar plate. The spore concentration was from 0.5 to 0.8×10⁷ spores per ml. 1 ml of DMSO and 10 μl of Syto 11 (dye stock solution in DMSO, Molecular Probes No. S-7573) were added to 9 ml of spore suspension. This was followed by staining at 30° C. for 2 h. Selection and application were carried out by means of a Becton Dickinson FacsVantage+Diva Option type instrument. First, a size selection was carried out in order to separate individual spores from aggregates and contaminations. These spores were then applied sorted according to their fluorescence (excitation=488 nm; emission=530 nm). The left shoulder of the Gauss curve of the fluorescence frequency distribution contained the mononuclear spores.

2) Preparation of Homonuclear Strains by Reducing the Number of Nuclei and Selection with FACS

To reduce the number of nuclei per spore, spore suspensions were treated with MNNG (N-methyl-N′-nitro-N-nitrosoguanidine) prior to selection, thus achieving a reduction in the number of nuclei by chemical mutagenesis.

For this, first a spore suspension containing 1×10⁷ spores/ml in Tris/HCl buffer, pH 7.0 was prepared. The spore suspension was admixed with MNNG at a final concentration of 100 μg/ml. The time of incubation in MNNG was chosen in such a way that the survival rate of the spores was approx. 5%. After incubation with MNNG, the spores were washed three times with 1 g/l Span 20 in 50 mM phosphate buffer pH 7.0 and sorted and selected by the method described under 1).

As an alternative, it was also possible to reduce the number of nuclei in the spores by using X-rays and UV rays, as described by Cerdá-Olmedo and Patricia Reau in Mutation Res., 9(1970), 369-384.

3) Preparation of Homonuclear Strains by Selection for Recessive Selection Markers

A suitable recessive selection marker for selection of homonuclear mycelia is, for example, the recessive selection marker pyrG. Wild-type strains of Blakeslea trispora are pyrG⁺. These strains are unable to grow in the presence of the pyrimidine analog 5-fluoroorotate (FOA), because they convert FOA to lethal metabolites via orotidine 5′-monophosphate decarboxylase. Genetically modified pyrG⁻-homonuclear Blakeslea lack the enzyme activity of orotidine 5′-monophosphate decarboxylase. Consequently, these pyrG⁻ strains are unable to utilize 5-fluoroorotate. Therefore, these strains grow in the presence of FOA and uracil. If the pyrG⁻ mutation and the foreign DNA insert are coupled on the nucleus of a mononuclear spore, this spore may form homonuclear recombinant fungal mycelium.

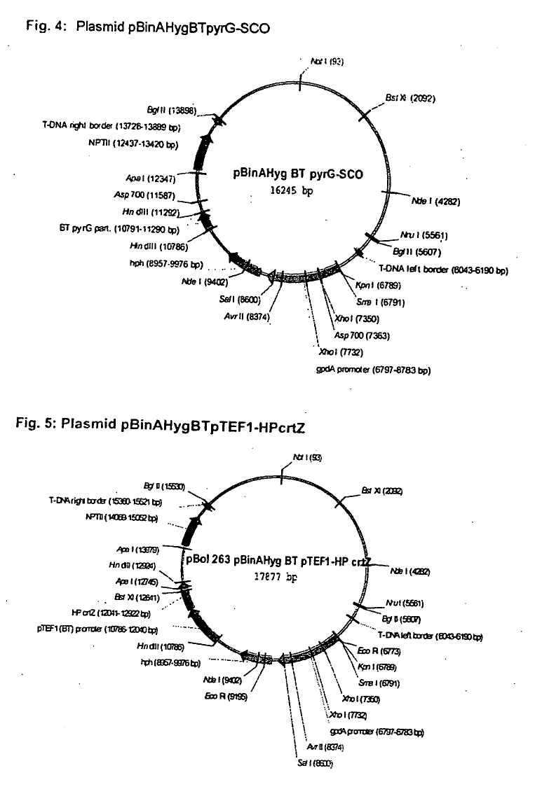

First, the plasmid pBinAHygBTpyrG-SCO (SEQ ID NO: 36, FIG. 4) was generated by inserting a fragment of pyrG (SEQ ID NO: 65) from Blakeslea trispora into pBinAHyg. Said plasmid was transformed into Blakeslea trispora and caused pyrG disruption there due to homologous recombination.

Homonuclear Blakeslea trispora GMO with the pyrG⁻ phenotype were selected as follows. Plating on MEP (30 g/l malt extract, 3 g/l peptone, pH 5.5, 18 g/l agar) containing 100 mg/l cefotaxime and 100 mg/l hygromycin for agrobacterium-mediated transformation of pBinAHygBTpyrG-SCO was carried out as described above.

The spores of the transformants were washed off with 10 ml of Tris-HCl 50 mM+0.1% Span20 per agar plate. The spore concentration was from 0.5 to 0.8×10⁷ spores per ml. The spores were then plated on FOA medium containing 100 mg/l cefotaxime and 100 mg/l hygromycin. FOA medium comprised, per liter, 20 g of glucose, 1 g of FOA, 50 mg of uracil, 200 ml of citrate buffer (0.5 M, pH 4.5) and 40 ml of trace salt solution according to Sutter, 1975, PNAS, 72:127). Homonuclear pyrG⁻ mutants exhibited growth on the uracil-containing FOA medium but no growth when plated on FOA medium without uracil. In the same way, homonuclear GMO were prepared from the Blakeslea trispora GMO described below for producing xanthophylls.

Alternatively, it is possible to plate the spores according to the protocol by Roncero et al. on medium comprising 5-carbon-5-deazariboflavin and, additionally, hygromycin (Roncero et al., 1984, Mutation Research, 125: 195-204). This enables homokaryotic cells of the genotype hyg^{R} and dar⁻ to be selected.

According to this principle, homokaryotic Blakeslea trispora strains with the phenotype hyg^{R} and dar⁻ are generated.

Exemplary Embodiments for Preparing Genetically Modified Organisms of Blakeslea trispora for Producing Carotenoids and Carotenoid Precursors.

The plasmids mentioned below were generated by the “overlap-extension PCR” method and by subsequent insertion of the amplification products into the pBinAHyg plasmid. The overlap-extension PCR method was carried out as described in Innis et al. (Eds) PCR protocols: a guide to methods and applications, Academic Press, San Diego. Transformation of the pBinAHyg derivatives and preparation of homonuclear genetically modified Blakeslea trispora strains were carried out as described above.

Genetically Modified Blakeslea trispora Strains for Producing Zeaxanthin

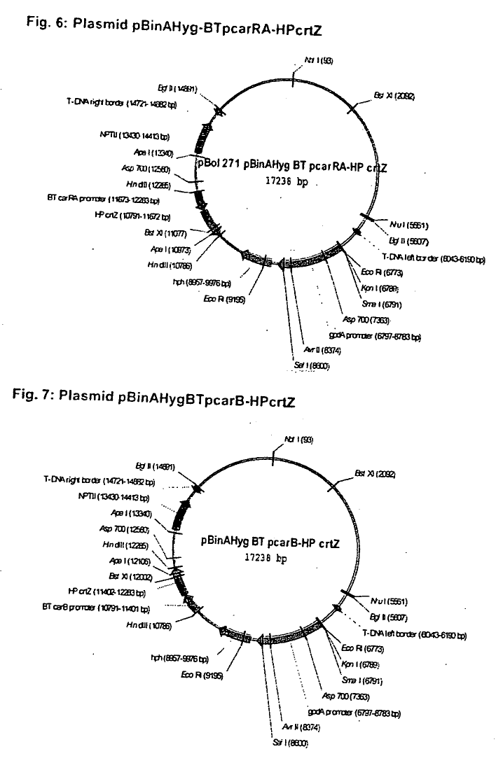

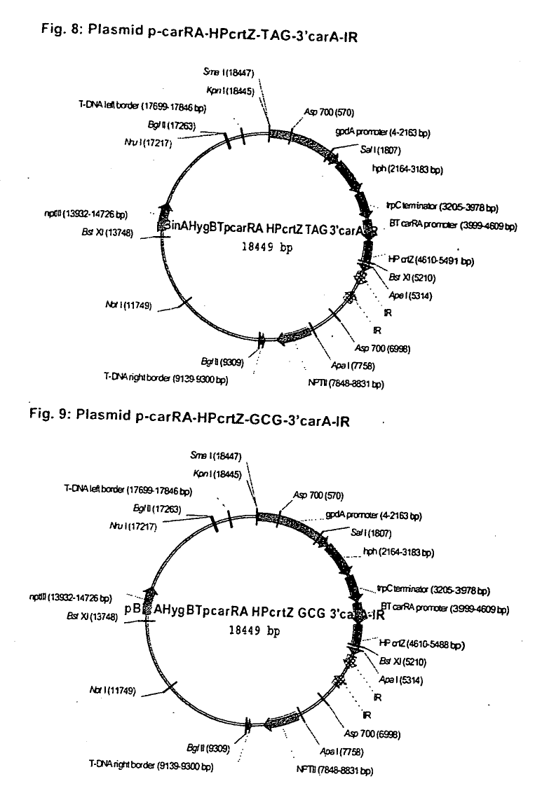

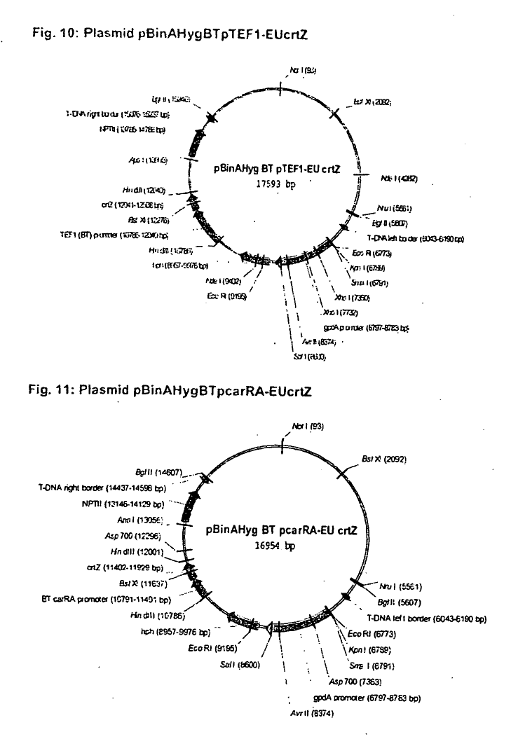

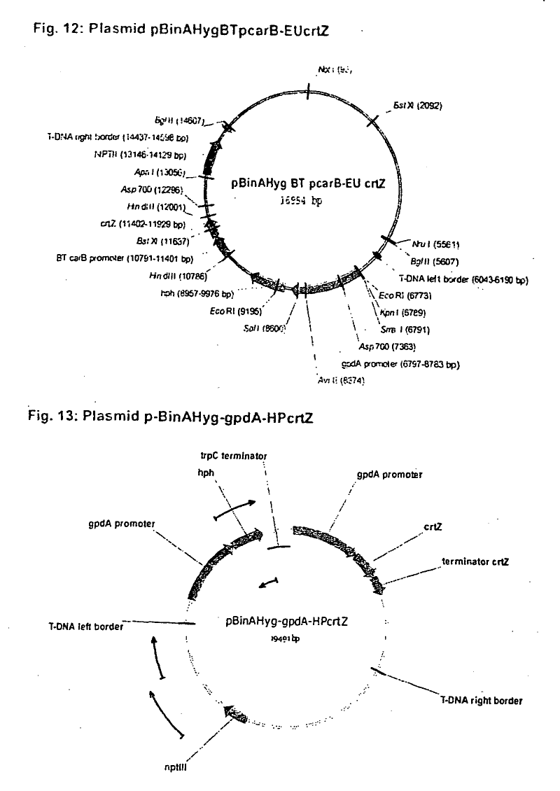

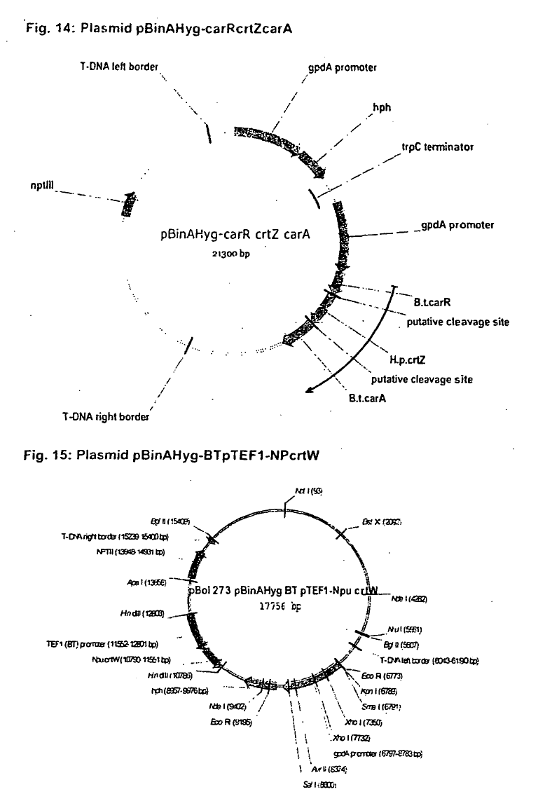

The following plasmids (pBinAHyg derivatives) were used for genetic modification of Blakeslea trispora for the production of zeaxanthin, and thus encode inter alia hydroxylases (crtZ):
ptef1-HPcrtZ, comprising the gene of the HPcrtZ hydroxylase (SEQ ID NO:70) from Haematococcus pluvialis Flotow NIES-144 (Accession No. AF162276) under the control of the Blakeslea trispora ptef1 promoter (Seq. pBinAHygBTpTEF1-HPcrtZ, SEQ ID NO:37, FIG. 5);
p-carRA-HPcrtZ, comprising the gene of the HPcrtZ hydroxylase from Haematococcus pluvialis Flotow NIES-144 under the control of the Blakeslea trispora pcarRA promoter (Seq. pBinAHygBTpcarRA-HPcrtZ, SEQ ID NO:38, FIG. 6);
p-carB-HPcrtZ, comprising the gene of the HPcrtZ hydroxylase from Haematococcus pluvialis Flotow NIES-144 under the control of the Blakeslea trispora pcarB promoter (Seq. pBinAHygBTpcarB-HPcrtZ, SEQ ID NO:39, FIG. 7);
p-carRA-HPcrtZ-TAG-3′carA-IR, comprising the gene of the HPcrtZ hydroxylase from Haematococcus pluvialis Flotow NIES-144 under the control of the Blakeslea trispora pcarRA promoter. An inverted repeat structure is located downstream of the hydroxylase gene, which structure is derived from the 3′ end of carA and the region downstream of carA (IR, SEQ ID NO:74, “Inverted Repeat 1” approx. 350 bp of carA, then approx. 200 bp “Loop” and then approx. 350 bp “Inverted Repeat 2”) (Seq. pBinAHyg-BTpcarRA-HPcrtZ-TAG-3′carA-IR, SEQ ID NO:40, FIG. 8);
p-carRA-HPcrtZ-GCG-3′carA-IR, comprising the gene of the HPcrtZ hydroxylase from Haematococcus pluvialis Flotow NIES-144 under the control of the Blakeslea trispora pcarRA promoter. The hydroxylase gene is fused to an inverted repeat structure which is derived from the 3′ end of carRA and the region downstream of carA (IR, SEQ ID NO:74, “Inverted Repeat 1” approx. 350 bp of carA, then approx. 200 bp “Loop” and then approx. 350 bp “Inverted Repeat 2”). Consequently, the derived fusion protein consists of the Haematococcus pluvialis hydroxylase and the carboxy terminus of Blakeslea trispora CarA (Seq. pBinAHyg-BTpcarRA-HPcrtZ-GCG-3′carA-IR, SEQ ID NO:41, FIG. 9).
p-tef1-EUcrtZ, comprising the gene of the EUcrtZ hydroxylase (SEQ ID NO:71) from Erwinia uredova 20D3 (Accession No. D90087) under the control of the ptef1 promoter (Seq. pBinAHygBTpTEF1-EUcrtZ, SEQ ID NO:42, FIG. 10);
p-carRA-EUcrtZ, comprising the gene of the EUcrtZ hydroxylase from Erwinia uredova 20D3 under the control of the Blakeslea trispora pcarRA promoter (Seq. pBinAHygBTpcarRA-EUcrtZ, SEQ ID NO:43, FIG. 11);
p-carB-EUcrtZ, comprising the gene of the EUcrtZ hydroxylase from Erwinia uredova 20D3 under the control of the Blakeslea trispora pcarB promoter (Seq. pBinAHygBTpcarB-EUcrtZ, SEQ ID NO:44, FIG. 12);
p-gpdA-HPcrtZ-t-crtZ, comprising the gene of the HPcrtZ hydroxylase from Haematococcus pluvialis Flotow NIES-144 under the control of the gpdA promoter and the t-crtZ terminator; i.e. of the sequence section downstream of crtZ from Haematococcus pluvialis Flotow NIES-144 (SEQ ID NO:73) (Seq. pBinAHyg-gpdA-HPcrtZ-tcrtZ, SEQ ID NO:43, FIG. 13).
p-gpdA-BTcarR-HPcrtZ-BTcarA, comprising a gene fusion of genes of lycopine cyclase carR from Blakeslea trispora, of HPcrtZ hydroxylase from Haematococcus pluvialis Flotow NIES-144 and of the phytoene synthase carA from Blakeslea trispora and under the control of the Aspergillus nidulans gpdA promoter (Seq. pBinAHyg-carR_crtZ_carA, SEQ ID NO:46, FIG. 14).

Preparation of Genetically Modified Blakeslea trispora Strains for Producing Canthaxanthin

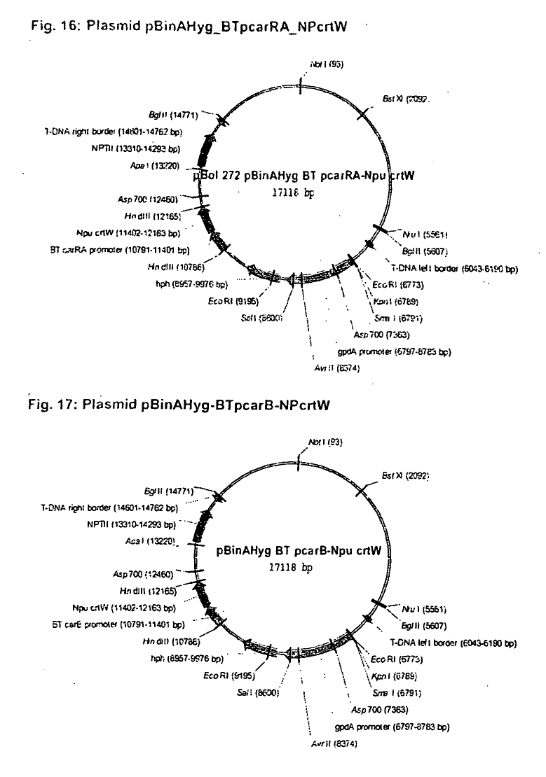

The following plasmids (pBinAHyg derivatives) were used for genetic modification of Blakeslea trispora for the production of canthaxanthin, and thus encode inter alia ketolases (crtW):
p-tef1-NPcrtW, comprising the gene of the NPcrtW ketolase (SEQ ID NO:72) from Nostoc punctiforme PCC73102 (ORF148, Accession No. NZ_AABC01000196) and under the control of the Blakeslea trispora ptef1 promoter (Seq. pBinAHygBTpTEF1-NpucrtW, SEQ ID NO:47, FIG. 15);
p-carRA-NPcrtW, comprising the gene of the NPcrtW ketolase from Nostoc punctiforme PCC73102 and under the control of the Blakeslea trispora pcarRA promoter (Seq. pBinAHygBTpcarRA-NpucrtW, SEQ ID NO:48, FIG. 16);
p-carB-NPcrtW, comprising the gene of the NPcrtW ketolase from Nostoc punctiforme PCC73102 and under the control of the Blakeslea trispora pcarB promoter (Seq. pBinAHygBTpcarB-NpucrtW, SEQ ID NO:49, FIG. 17).

Preparation of Genetically Modified Blakeslea trispora Strains for Producing Astaxanthin

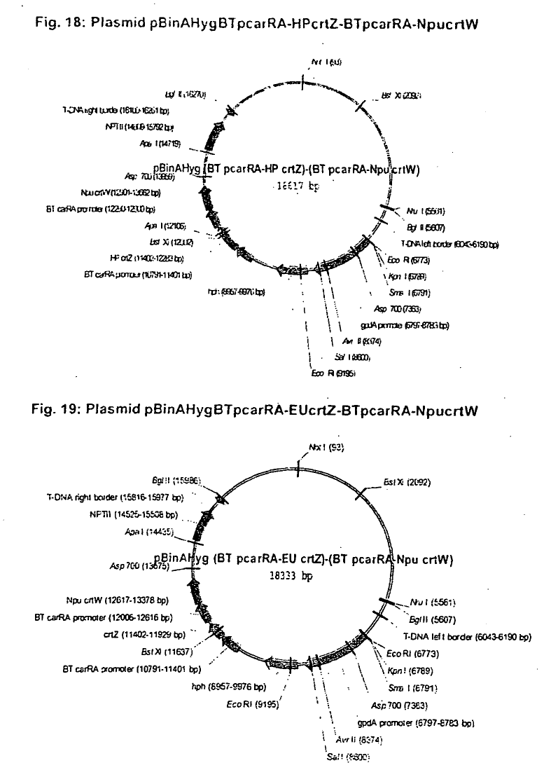

The following plasmids (pBinAHyg derivatives) were used for genetic modification of Blakeslea trispora for producing astaxanthin, i.e. encode inter alia hydroxylases (crtZ) and ketolases (crtW):
p-carRA-HPcrtZ-pcarRA-NPcrtW, comprising the gene of the HPcrtZ hydroxylase from Haematococcus pluvialis Flotow NIES-144 and the gene of the NPcrtW ketolase from Nostoc punctiforme PCC73102 (ORF148, Accession No. NZ-AABC01000196), both in each case under the control of the Blakeslea trispora pcarRA promoter (Seq. pBinAHygBTpcarRA-HPcrtZ-BTpcarRA-NpucrtW, SEQ ID NO:50, FIG. 18);
p-carRA-EUcrtZ-pcarRA-NPcrtW, comprising the gene of the EUcrtZ hydroxylase from Erwinia uredova 20D3 (Accession No. D90087) and the gene of the NPcrTW ketolase from Nostoc punctiforme PCC73102, both in each case under the control of the Blakeslea trispora pcarRA promoter (Seq. pBinAHygBTpcarRA-EUcrtZ-BTpcarRA-NpucrtW, SEQ ID NO:51, FIG. 19).

Cloning and Sequence Analysis of Genes and Promoters which may be Utilized by Way of Example for Genetic Modification of Blakeslea trispora.

Cloning and sequencing of various Blakeslea trispora genes and promoters are described by way of example below.

Cloning and Sequence Analysis of ptef1

Blakeslea trispora p-tef was cloned on the basis of a sequence, previously published in GenBank, of the structural gene of Blakeslea trispora translation elongation factor 1-α (AF157235). Starting from the sequence entry AF157235 primers were selected for inverted PCR in order to amplify and sequence the promoter region upstream of said structural gene. In the inverted nested PCR of 200 ng of XhoI-cleaved and circularized genomic DNA of Blakeslea trispora ATCC14272, a 3000-bp fragment was obtained in the following reaction mixture: template DNA (1 μg of genomic DNA of Blakeslea trispora ATCC 14272) primers MAT344 5′-GGCGTACTTGAAGGAACCCTTACCG-3′ (SEQ ID NO: 63) and MAT 345 5′-ATTGATGCTCCCGGTCACCGTGATT-3′ (SEQ ID NO: 64), 0.25 μM each, 100 μM dNTP, 10 μl of Herculase polymerase buffer 10×, 5 U of Herculase (addition at 85° C.), H₂O ad 100 μl. The PCR profile was as follows: 95° C., 10 min (1 cycle); 85° C., 5 min (1 cycle); 60° C., 30 s, 72° C., 60 s, 95° C., 30 s (30 cycles); 72° C., 10 min (1 cycle). The sequence section upstream of the putative start codon of the tef1 gene in the 3000-bp fragment was referred to as ptef1 promoter.

Cloning, Sequence Analysis of the Gene of HMG-CoA Reductase from Blakeslea trispora

First, the cosmid vector pANsCos1 was used for preparing a gene library of Blakeslea trispora ATCC 14272, Mating Type (−). The vector was linearized by cleavage with XbaI and then dephosphorylated. Further cleavage with BamHI generated the insertion site into which the Blakeslea trispora genomic DNA, partially cleaved with Sau3AI and dephosphorylated, was ligated. The cosmids produced in this way were subsequently packaged in vitro and transferred into Escherichia coli.

On the basis of the known sequence of a fragment of the Blakeslea trispora gene encoding HMG-CoA reductase (Eur. J. Biochem 220, 403-408 (1994)), a 315-bp DNA probe was prepared by the following PCR. Reaction mixture: 1 μg of genomic DNA of Blakeslea trispora ATCC 14272, primers MAT314 5′-CCGATGGCGACGACGGAAGGTTGTT-3′ [SEQ ID NO: 79] and MAT315 5′-CATGTTCATGCCCATTGCATCACCT-3′ [SEQ ID NO: 80], 0.25 μM each, 100 μM dNTP, 10 μl of Herculase polymerase buffer 10×, 5 U of Herculase (addition at 85° C.), H₂O ad 100 μl. The PCR profile was as follows: 95° C., 10 min (1 cycle); 85° C., 5 min (1 cycle); 58° C., 30 s, 72° C., 30 s, 95° C., 30 s (30 cycles) ; 72° C., 10 min (1 cycle).

This DNA probe was used for screeing the cosmid gene library. A clone whose cosmid hybridized with said DNA probe was identified. The insert of this cosmid was sequenced. The DNA sequence comprised a section which was assigned to the gene of an MHG-CoA reductase [SEQ ID NO 75].

Cloning and Sequence Analysis of carB

(carB=Blakeslea trispora phytoene desaturase gene) The degenerated primers MAT182 5′-GCNGARGGNATHTGGTA-3′ (SEQ ID 52) and MAT192 5′-TCNGCNAGRAADATRTTRTG-3′ (SEQ ID 53) were derived from comparing the peptide sequences of phytoene desaturases and comparing the corresponding DNA sequences of Phycomyces blakesleeanus, Cercospora nicotianae, Phaffia rhodozyma and Neurospora crassa. The PCR was carried out in 100 μl reaction mixtures. These comprised 200 ng of genomic DNA of Blakeslea trispora ATCC14272, 1 μM MAT182, 1 μM MAT192, 100 μM dNTP, 10 μl of Pfu polymerase buffer 10×, 2.5 U of Pfu polymerase (addition at 85° C.), H₂O ad 100 μl.

The PCR profile was as follows: 95° C., 10 min (1 cycle); 85° C., 5 min (1 cycle); 40° C., 30 s, 72° C, 30 s, 95° C., 30 s (35 cycles); 72° C., 10 min (1 cycle).

This resulted in a 358-bp fragment whose derived peptide sequence is similar to the phytoene desaturase sequences. The method of inverted PCR (Innis et al. in PCR protocols: a guide to methods and applications. 1990. pp. 219-227) was used for amplifying, cloning and sequencing, according to the principle of chromosome walking, the gene regions upstream and downstream of the 350-bp fragment as follows:
(i) a 1.1 kbp fragment, by PCR with the primers MAT219 5′-AAGTGACACCGGTTACACGCTTGTCTT-3′ (SEQ ID 54) and MAT 220 5′-GCTTATCACCATCTGTTACCTCCTTGC-3′ (SEQ ID 55), obtained from 200 ng of EcoRI-cleaved and circularized genomic DNA of Blakeslea trispora ATCC14272, 0.25 μM MAT219, 0.25 μM MAT220, 100 μM dNTP, 10 μl of Herculase polymerase buffer 10×, 5 U of Herculase (addition at 85° C.), H₂O ad 100 μl. The PCR profile as as follows: 95° C., 10 min (1 cycle); 85° C., 5 min (1 cycle); 60° C., 30 s, 72° C., 60 s, 95° C., 30 s (30 cycles); 72° C., 10 min (1 cycle),
(ii) a 2.9 kbp fragment, by PCR with the primers MAT219 and MAT220, obtained from 200 ng of XbaI cleaved and circularized genomic DNA Blakeslea trispora ATCC14272, 0.25 μM MAT219, 0.25 μM MAT220, 100 μM dNTP, 10 μl of Herculase polymerase buffer 10×, 5 U of Herculase (addition at 85° C.), H₂O ad 100 μl. The PCR profile was as follows: 95° C., 10 min (1 cycle); 85° C., 5 min (1 cycle); 60° C., 30 s, 72° C., 3 min, 95° C., 30 s (30 cycles); 72° C., 10 min (1 cycle).

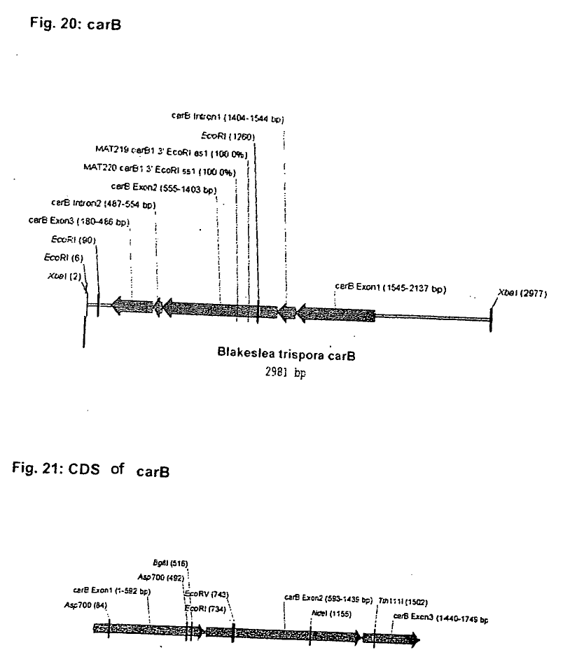

FIG. 20 [SEQ ID NO 77] depicts diagrammatically the cloned sequence section. Sequencing was carried out in strand and counterstrand orientation, using the cloned fragments and the PCR products. FIG. 21 [SEQ ID NO 78] depicts the sequence of the cloned sequence section.

Sequence Comparisons

The nucleotide sequence of carB and the peptide sequence of the derived protein CarB were compared with the known sequences of related proteins. The sequences were compared using the GAP and BESTFIT programs.

CarB—Identical Aminoacyl Residues According to GAP

Program settings:

| | |
|---|---|
| Gap weight: | 8 |
| Length weight: | 2 |
| Average match: | 2.912 |
| Average mismatch: | −2.003 |

The following values, in %, of amino acid correspondence to CarB of Blakeslea trispora ATCC14272 were found:

| | |
|---|---|
| Phycomyces blakesleeanus: | 72.491 |
| Phaffia rhodozyma: | 50.460 |
| Neurospora crassa: | 47.943 |
| Cercospora nicotianae: | 47.740 |

CarB—Identical Aminoacyl Residues According to BESTFIT

Program settings:

| | |
|---|---|
| Gap weight: | 8 |
| Length weight: | 2 |
| Average match: | 2.912 |
| Average mismatch: | −2.003 |

The following values, in %, of amino acid correspondence to CarB of Blakeslea trispora ATCC14272 were found:

| | |
|---|---|
| Phycomyces blakesleeanus: | 73.380 |
| Phaffia rhodozyma: | 53.175 |
| Neurospora crassa: | 51.896 |
| Cercospora nicotianae: | 50.791 |

carB—Identical Bases According to GAP

Program settings:

| | |
|---|---|
| Gap weight: | 50 |
| Length weight: | 3 |
| Average match: | 10.000 |
| Average Mismatch: | 0.000 |

The following values, in %, of base correspondence to CarB of Blakeslea trispora ATCC14272 were found:

| | |
|---|---|
| Phycomyces blakesleeanus: | 64.853 |
| Cercospora nicotianae: | 50.143 |
| Phaffia rhodozyma: | 43.179 |
| Neurospora crassa: | 42.130 |

carB—Identical Bases According to BESTFIT

Program settings:

| | |
|---|---|
| Gap weight: | 50 |
| Length weight: | 3 |
| Average match: | 10.000 |
| Average mismatch: | −9.000 |

The following values, in %, of base correspondence to CarB of Blakeslea trispora ATCC14272 were found:

| | |
|---|---|
| Phycomyces blakesleeanus: | 68.926 |
| Phaffia rhodozyma: | 62.403 |
| Neurospora crassa: | 60.230 |
| Cercospora nicotianae: | 56.884 |

Cloning for carB Expression

In order to clone and express Blakeslea trispora carB, the possible protein sequences were derived in six reading frames from the above-described cloned sequence section from Blakeslea trispora. These protein sequences were compared with the sequences of the phytoene desaturates from Phycomyces blakesleeanus, Phaffia rhodozyma, Neurospora crassa, Cercospora nicotianae. On the basis of the sequence comparison, three exons were identified in the cloned sequence section of the Blakeslea trispora genomic DNA, which, put together, result in a coding region whose derived gene product has, over its entire length, 72.7% identical aminoacyl residues with the CarB phytoene desaturase of Phycomyces blakesleeanus. This sequence section comprising three possible exons and two possible introns was therefore referred to as gene carB. In order to check the predicted gene structure, the coding sequence of Blakeslea trispora carB was generated by means of PCR using Blakeslea trispora cDNA as template and the primers Boll425 5′-AGAGAGGGATCCTTAAATGCGAATATCGTTGC-3′ (SEQ ID 56) and Boll426 5′-AGAGAGGGATCCATGTCTGATCAAAAGAAGCA-3′ (SEQ ID 57). The DNA fragment obtained was sequenced. The location of exons and introns was confirmed by comparing the cDNA with the genomic carB DNA. FIG. 21 depicts diagrammatically the coding sequence of carB. For expression of carB in Escherichia coli, first the NdeI cleavage site in carB was removed by the overlap extension PCR method and an NdeI cleavage site was introduced at the 5′ end of the gene and a BamHI cleavage site was introduced at the 3′ end. The DNA fragment obtained was ligated with the vector pJOE2702. The plasmid obtained was referred to as pBT4 and cloned together with pCAR-AE into Escherichia coli XL1-Blue. Expression was induced with rhamnose. The enzyme activity was detected by way of detecting lycopine synthesis via HPLC. The cloning steps are described below:

PCR 1.1:

Approx. 0.5 μg of Blakeslea trispora cDNA, 0.25 μM MAT350 5′-ACTTTATTGGATCCTTAAATGCGAATATCGTTGCTGC-3′ (SEQ ID 58), 0.25 μM MAT244 5′-GTTCCAATTGGCCACATGAAGAGTAAGACAGGAAACAG-3′ (SEQ ID 59), 100 μM dNTP, 10 μl of Pfu polymerase buffer (10×), 2.5 U of Pfu polymerase (addition at 85° C., “hot start”) and H₂O ad 100 μL.

Temperature Profile:

1. 95° C. 10 min, 2. 85° C. 5 min, 3. 40° C. 30 s, 4. 72° C. 1 min 30 s, 5. 95° C. 30 s, 6. 50° C. 30 s, 7. 72° C. 1 min 30 s, 8. 95° C. 30 s, 9. 72° C. 10 min

Cycles: (1-2.) 1×, (3-5.) 5×, (6-8.) 25×, (9.) 1×

PCR 1.2:

Approx. 0.5 μg of Blakeslea trispora cDNA, 0.25 μM MAT243 5′-CCTGTCTTACTCTTCATGTGGCCAATTGGAACCAACAC-3′ (SEQ ID 60), 0.25 μM MAT353 5′-CTATTTTAATCATATGTCTGATCAAAAGAAGCATATTG-3′ (SEQ ID 61), 100 μM dNTP, 10 μl of Pfu polymerase buffer (10×), 2.5 U of Pfu polymerase (addition at 85° C., “hot start”) and H₂O ad 100 μL.

Temperature Profile:

1. 95° C. 10 min, 2. 85° C. 5 min, 3. 40° C. 30 s, 4. 72° C. 1 min 30 s, 5. 95° C. 30 s, 6. 50° C. 30 s, 7. 72° C. 1 min 30 s, 8. 95° C. 30 s, 9. 72° C. 10 min

Cycles: (1-2.) 1×, (3-5.) 5×, (6-8.) 25×, (9.) 1×

Purification of the PCR Fragments from PCR 1.1, 1.2

For this purpose, PCR 2 was carried out to prepare the coding sequence of Blakeslea trispora carB for cloning into pJOE2702:

Approx. 50 ng of PCR 1.1 product and approx. 50 ng of PCR 1.2 product, with 0.25 μM MAT350 (5′-ACTTTATTGGATCCTTAAATGCGAATATCGTTGCTGC-3′ SEQ ID NO 58), 0.25 μM MAT353 (5′-CTATTTTAATCATATGTCTGATCAAAAGAAGCATATTG-3′ SEQ ID NO 61), 100 μM dNTP, 10 μL of Pfu polymerase buffer (10×), 2.5 U of Pfu polymerase (addition at 85° C., “hot start”) and H₂O ad 100 μL.

Temperature Profile:

1. 95° C. 10 min, 2. 85° C. 5 min, 3. 59° C. 30 s, 4. 72° C. 2 min, 5. 95° C. 30 s, 6. 72° C. 10 min

Cycles: (1-2.) 1×, (3-5.) 22×, (6.) 1×

Subsequently, the fragment obtained (˜1.7 kbp) was purified, followed by ligation into the vector pPCR-Script-Amp, cloning into Escherichia coli XL1-Blue, sequencing of the insert, cleavage with NdeI and BamHI and ligation into pJOE2702. The plasmid obtained was referred to as pBT4.

Characterization and Detection of the Enzyme Activity of CarB (Phytoene Desaturase)

The gene product derived from carB was referred to as CarB. CarB has the following properties, based on peptide sequence analysis:

| | |
|---|---|
| Length: | 582 aminoacyl residues |
| Molecular mass: | 66470 |
| Isoelectric point: | 6.7 |
| Catalytic activity: | Phytoene desaturase |
| Reactant: | Phytoene |
| Product: | Lycopene |
| EC number: | EC 1.14.99- |

The enzyme activity was detected in vivo. Transfer of the plasmid (pCAR-AE) into Escherichia coli XL1-Blue produces the strain Escherichia coli XL1-Blue (pCAR-AE). This strain synthesizes phytoene. An additional transfer of the pBT4 plasmid into Escherichia coli XL1-Blue produces the strain Escherichia coli XL1-Blue (pCAR-AE) (pBT4). Since an enzymicly active phytoene desaturase is formed starting from carB, this strain produces lycopene.

The plasmids pCAR-AE and pBT4 were therefore transferred into Escherichia coli. The carotenoids were extracted from the cells grown in liquid culture and characterized (cf. above).

HPLC analysis revealed that the Escherichia coli XL1-Blue (pCAR-AE) strain produces phytoene and the Escherichia coli XL1-Blue (pCAR-AE) (pBT4) strain produces lycopene. Consequently, CarB has the enzyme activity of a phytoene desaturase.

Preparation of Genetically Modified Blakeslea trispora Strains for Producing Phytoene

The preparation of genetically modified organisms for producing phytoene is described by way of example below.

Vector pBinAHygΔcarB for Generating carB⁻ Mutants of Blakeslea trispora

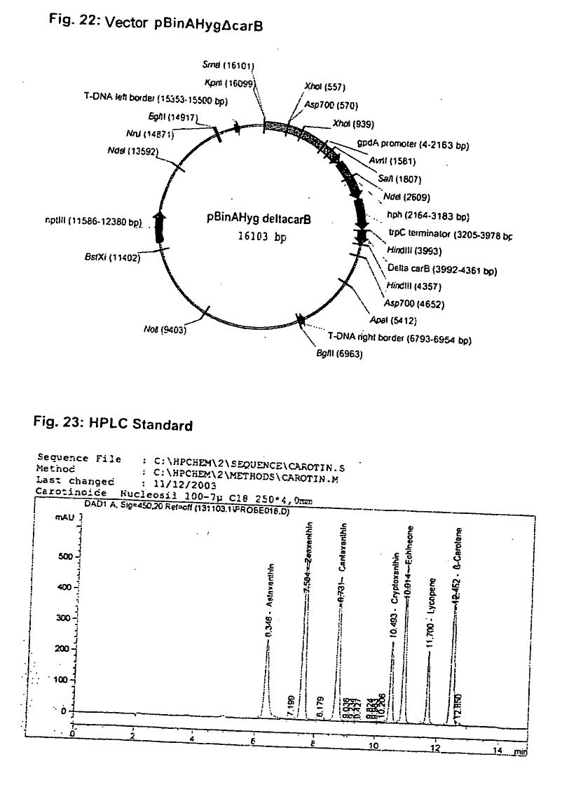

The vector pBinAHygΔcarB (SEQ. ID. NO:62, FIG. 22) was constructed to delete carB in Blakeslea trispora. The precursor of pBinAHygΔcarB is pBinAHyg (SEQ. ID. NO:3, FIG. 2) which was constructed as follows: The gpdA-hph cassette was isolated as BglII/HindIII fragment from the plasmid pANsCos1 (SEQ ID. NO:4, FIG. 1, Osiewacz, 1994, Curr. Genet. 26:87-90) and ligated into the BamHI/HindIII-opened binary plasmid pBin19 (Bevan, 1984, Nucleic Acids Res. 12:8711-8721). The vector obtained in this way was referred to as pBinAHyg and comprises the E. coli hygromycin resistance gene (hph) under the control of the gpd promoter and the trpC terminator from Aspergillus nidulans and the appropriate border sequences required for the Agrobacterium DNA transfer.

The carB coding sequence was amplified by means of PCR using the primers MAT350 and MAT353 and the following parameters:

50 ng of pBT4 with 0.25 μM MAT350 (5′-ACTTTATTGGATCCTTAAATGCGAATATCGTTGCTGC-3′; SEQ ID NO 58), 0.25 μM MAT353 (5′-CTATTTTAATCATATGTCTGATCAAAAGAAGCATATTG-3′; SEQ ID NO 61), 100 μM dNTP, 10 μl of Pfu polymerase buffer, 2.5 U of Pfu polymerase (addition at 85° C., “hot start”) and H₂O to 100 μl

Temperature Profile:

1. 95° C. 10 min, 2. 85° C. 5 min, 3. 58° C. 30s, 4. 72° C. 2 min, 5. 95° C. 30 s, 6. 72° C. 10 min.

Cycles: (1.-2.) 1×, (3-5.) 30×, (6.) 1×

The fragment obtained (˜1.7 kbp) was subsequently purified, followed by cleavage with HindIII, further purification of the 364 bp HindIII fragment carB, followed by cleavage of pBinAHyg with HindIII, ligation of the 364 bp HindIII fragment carB into pBinAHyg, transformation of the vector into Escherichia coli and isolation of the construct and referred to as pBinAHygΔcarB, as described above. Alternatively, partial cleavage with HindIII was carried out and a larger carB HindIII fragment was cloned into pBinAHyg to produce pBinAHygΔcarB.

Generation of carB⁻ Mutants of Blakeslea trispora

The pBinAHygΔcarB plasmid was first transferred into the Agrobacterium strain LBA 4404, for example by electroporation (cf. above). The plasmid was subsequently transferred from Agrobacterium tumefaciens LBA 4404 in Blakeslea trispora ATCC 14272 and in Blakeslea trispora ATCC 14271 (cf. above). Successful detection of the gene transfer into Blakeslea trispora was carried out via polymerase chain reaction according to the following protocol:

approx. 0.5 ug of DNA from Blakeslea trispora ATCC 14272 carB⁻ or ATCC 14271 carB⁻ was reacted with 0.25 μM primer hph forward (5′-CGATGTAGGAGGGCGTGGATA-3′; SEQ ID NO 5), 0.25 μM primer hph reverse (5′-GCTTCTGCGGGCGATTTGTGT-3′; SEQ ID NO 6), 100 μM dNTP, 10 μL of Herculase polymerase buffer, 2.5 U of Herculase DNA polymerase (addition at 85° C., “hot start”) and H₂O to 100 μl.

Temperature Profile:

1. 95° C. 10 min, 2. 85° C. 5 min, 3. 58° C. 1 min, 4. 72° C. 1 min, 5. 94° C. 1 min, 6. 72° C. 10 min.

Cycles: (1.-2.) 1×, (3-5.) 30×, (6.) 1×

It was attempted to amplify the Agrobacterium kanamycin resistance gene as a negative control. For this purpose, the following PCR conditions were used:

approx. 0.5 μg of DNA from Blakeslea trispora ATCC 14272 carB⁻ and ATCC 14271 carB⁻ was reacted with 0.25 μM primer nptIII forward (5′-TGAGAATATCACCGGAATTG-3′; SEQ ID NO 7), 0.25 μM primer nptIII reverse (5′-AGCTCGACATACTGTTCTTCC-3′; SEQ ID NO 8), 100 μM dNTP, 10 μL of Herculase polymerase buffer, 2.5 U of Herculase DNA polymerase (addition at 85° C., “hot start”) and H₂O to 100 μl.

Temperature Profile:

1. 95° C. 10 min, 2. 85° C. 5 min, 3. 58° C. 1 min, 4. 72° C. 1 min, 5. 94° C. 1 min, 6. 72° C. 10 min

Cycles: (1-2.) 1×, (3-5.) 30×, (6.) 1×

Production of Carotenoids and Carotenoid Precursors by Blakeslea trispora

The carotenoids zeaxanthin, canthaxanthin, astaxanthin and phytoene were produced by fermenting the corresponding genetically modified Blakeslea trispora (+) and (−) strains, detecting the carotenoid produced by means of HPLC analysis and isolating it.

The liquid medium for producing carotenoids comprised, per liter: 19 g of cornflour, 44 g of soybean flour, 0.55 g of KH₂PO₄, 0.002 g of thiamine hydrochloride, 10% sunflower oil. The pH was adjusted to 7.5 with KOH.

To produce the carotenoids, shaker flasks were inoculated with spore suspensions of (+) and (−) strains of the Blakeslea trispora GMO. The shaker flasks were incubated at 26° C. and 250 rpm for 7 days. Alternatively, trisporic acids were added to mixtures of the strains after 4 days, followed by 3 more days of incubation. The final concentration of the trisporic acids was 300-400 μg/ml.

Extraction and Analysis Extraction:

1. Removal of 10 ml of culture suspension

2. Centrifugation, 10 min, 5000×g

3. Discarding of the supernatant

4. Resuspension of the pellet in 1 ml of tetrahydrofuran (THF) by vortexing

5. Centrifugation, 5 min, 5000×g

6. Removal of the THF phase

7. Repetition of steps 4.-6. (2×)

8. Pooling of the THF phases

9. Centrifugation of the pooled THF phases at 20000×g for 5 min in order to remove residual aqueous phase.

Analysis

Phytoene Measurement by Means of HPLC

| Column: | ZORBAX Eclipse XDB-C8, 5 um, 150 * 4.6 mm | | | |
|---|---|---|---|---|
| Temperature: | 40° C. | | | |
| Flow rate: | 0.5 ml/min | | | |
| Injection volume: | 10 μl | | | |
| Detection: | UV 220 nm | | | |
| Stop time: | 12 min | | | |
| Post run time: | 0 min | | | |
| Maximum pressure: | 350 bar | | | |
| Eluent A: | 50 mM NaH₂PO₄, pH 2.5 with | | | |
| | perchloric acid | | | |
| Eluent B: | Acetonitrile | | | |
| Gradient: | Time [min] | A [%] | B [%] | Flow [ml/min] |
| | 0 | 50 | 50 | 0.5 |
| | 12 | 50 | 50 | 0.5 |

Extracts of the fermentation broth were used as matrix. Prior to HPLC, each sample was filtered through a 0.22 μm filter. The samples were kept cool and protected from light. In each case 50-1000 mg/l were weighed and dissolved in THF for calibration. The standard used was phytoene which has a retention time of 7.7 min under the given conditions.

Measurement of Lycopene, β-Carotene, Echinenone, Canthaxanthin, Cryptoxanthin, Zeaxanthin and Astaxanthin by Means of HPLC

| Column: | Nucleosil 100-7 C18, 250 * 4.0 mm | | | |
|---|---|---|---|---|
| | (Macherey & Nagel) | | | |
| Temperature: | 25° C. | | | |
| Flow rate: | 1.3 ml/min | | | |
| Injection volume: | 10 μl | | | |
| Detection: | 450 nm | | | |
| Stop time: | 15 min | | | |
| Post run time: | 2 min | | | |
| Maximum pressure: | 250 bar | | | |
| Eluent A: | 10% acetone, 90% H₂O | | | |
| Eluent B: | Acetone | | | |
| Gradient: | Time [min] | A [%] | B [%] | Flow [ml/min] |
| | 0 | 30 | 70 | 1.3 |
| | 10 | 5 | 95 | 1.3 |
| | 12 | 5 | 95 | 1.3 |
| | 13 | 30 | 70 | 1.3 |

Extracts of the fermentation broth were used as matrix. Prior to HPLC, each sample was filtered through a 0.22 μm filter. The samples were kept cool and protected from light. In each case 10 mg were weighed and dissolved in 100 ml of THF for calibration. The following carotenoids with the following retention times were used as standard: β-carotene (12.5 min), lycopene (11.7 min), echinenone (10.9 min), cryptoxanthin (10.5 min), canthaxanthin (8.7 min)., zeaxanthin (7.6 min) and astaxanthin (6.4 min) [see FIG. 23].

Production of Zeaxanthin by Genetically Modified Blakeslea trispora Strains

Production of zeaxanthin by genetically modified organisms (GMO) of Blakeslea trispora is described by way of example below.

The vector pBinAHygBTpTEF1-HPcrtZ was transferred into Blakeslea trispora by Agrobacterium-mediated transformation (see above). A hygromycin-resistant clone was isolated and transferred to a potato-glucose agar plate (Merck KGaA, Darmstadt, Germany).

Starting from this plate, a spore suspension was prepared after three days of incubation at 26° C. A 250 ml Erlenmeyer flask without baffles and comprising 50 ml of growth medium (47 g/l cornflour, 23 g/l soybean flour, 0.5 g/l KH₂PO₄, 2.0 mg/l thiamine-HCl, pH adjusted to 6.2-6.7 with NaOH before sterilization) was inoculated with 1×10⁵ spores. This preculture was incubated at 26° C. and 250 rpm for 48 hours. For the main culture, a 250 ml Erlenmeyer flask without baffles and comprising 40 ml of production medium was inoculated with 4 ml of the preculture and incubated at 26° C. and 150 rpm for 8 days. The production medium comprised 50 g/l glucose, 2 g/l caseine acid hydrolysate, 1 g/l yeast extract, 2 g/l L-asparagine, 1.5 g/l KH₂PO₄, 0.5 g/l MgSO₄×7 H₂O, 5 mg/l thiamine-HCl, 10 g/l Span20, 1 g/l Tween 80, 20 g/l linoleic acid, 80 g/l corn steep liquor. After 72 hours, kerosene was added at a final concentration of 40 g/l. After harvesting the cultures, the remaining culture volume of approximately 35 ml was increased to 40 ml with water. Subsequently, the cells were disrupted in a high pressure homogenizer, type Micron Lab 40, APV Gaulin, 3× at 1500 bar.

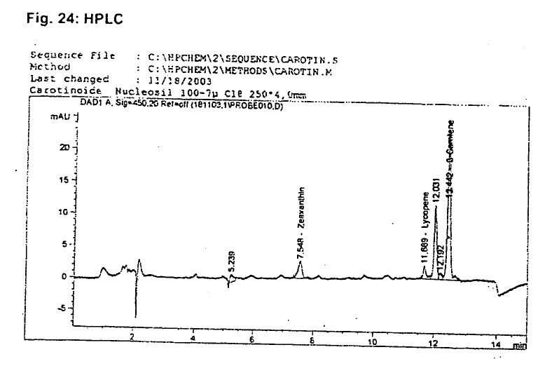

The suspension comprising the disrupted cells was admixed with 35 ml of THF and incubated with shaking at 250 rpm and RT in the dark for 60 min. Then 2 g of NaCl were added and the mixture was incubated with shaking once more. The extraction mixture was then centrifuged at 5000×g for 10 min. The colored THF phase was removed and the cell mass was completely colorless. The THF phase was concentrated to 1 ml in a rotary evaporator at 30 mbar and 30° C. and then taken up again in 1 ml of THF. After centrifugation at 20000×g for 5 min, an aliquot of the upper phase was removed and analyzed by HPLC (FIG. 24, FIG. 23).

1. A method for producing a genetically modified organism of the Blakeslea genus, which method comprises the following steps
(i) transformation of at least one of the cells, and
(ii) selection and cultivation of the genetically modified cell or cells.

2. The method according to claim 1, wherein the cells are from fungi of the Blakeslea trispora species.

3. The method according to claim 1, wherein a vector or free nucleic acids are used in the transformation of step (i).

4. The method according to claim 3, wherein the vector employed in the transformation is integrated into the genome of at least one of the cells.

5. The method according to claim 4, wherein the vector employed in the transformation comprises a promoter and/or a terminator.

6. The method according to claim 3, wherein a vector comprising the a gpd, pcarB, pcarRA and/or ptef1 promoter and/or a trpC terminator is employed in the transformation.

7. The method according to claim 3, wherein a vector comprising a resistance gene is employed in the transformation.

8. The method according to claim 7, wherein the vector employed in the transformation comprises a hygromycin resistance gene (hph).

9. The method according to claim 6, wherein the gpd promoter comprises the sequence SEQ ID NO: 1.

10. The method according to claim 6, wherein the trpC terminator comprises the sequence SEQ ID NO: 2.

11. The method according to claim 6, wherein the ptef1 promoter comprises the sequence SEQ ID NO: 35.

12. The method according to claim 6, wherein the gpd promoter and the trpC terminator are derived from Aspergillus nidulans.

13. The method according to claim 3, wherein the vector comprises the sequence SEQ ID NO: 3.

14. The method according to claim 1, wherein the transformation is carried out using agrobacteria, conjugation, chemicals, electroporation, bombardment with DNA-loaded particles, protoplasts or microinjection.

15. The method according to claim 1, wherein a mutagenic agent is employed in the homokaryotic conversion of step (ii).

16. The method according to claim 15, wherein the mutagenic agent employed is N-methyl-N′-nitronitrosoguanidine (MNNG), UV radiation or X rays.

17. The method according to claim 1, wherein the selection is carried out by labeling and/or selecting the mononuclear cells.

18. The method according to claim 1, wherein 5-carbon-5-deazariboflavin (darf) and hygromycin (hyg) or 5-fluororotate (FOA) and uracil and hygromycin are employed in the selection.

19. The method according to claim 3, wherein the vector employed in the transformation includes genetic information for producing carotenoids or their precursors.

20. The method according to claim 3, wherein the vector employed in the transformation includes genetic information for producing carotenes or xanthophylls.

21. The method according to claim 3, wherein the vector employed in the transformation includes genetic information for producing astaxanthin, zeaxanthin, echinenone, β-cryptoxanthin, andonixanthin, adonirubin, canthaxanthin, 3-hydroxyechinenone, 3′-hydroxyechinenone, lycopene, β-carotene, α-carotene, lutein, bixin, phytofluene or phytoene.

22. The method according to claim 3, wherein the vector employed in the transformation is designed so as to introduce the genetic information comprised therein into the Blakeslea trispora genome.

23. The method according to claim 3, wherein the vector employed in the transformation comprises genetic information displaying a ketolase activity and/or a hydroxylase activity after expression.

24. The method according to claim 23, wherein the vector employed in the transformation comprises SEQ ID NO: 70 or SEQ ID NO: 71 or SEQ ID NO: 76 and/or SEQ ID NO: 72.

25. The method according to claim 23, wherein the vector employed in the transformation has a sequence selected from the group consisting of SEQ ID NOs: 37-51.

26. The method according to claim 3, wherein the vector employed in the transformation is designed so that the genetic information comprised therein is switched off in the cell.

27. The method according to claim 3, wherein the transformation results in the switching off of a_phytoene desaturase gene.

28. The method according to claim 27, wherein the vector employed in the transformation comprises SEQ ID NO: 69.

29. The method according to claim 27, wherein the vector employed in the transformation comprises the sequence SEQ ID NO: 62.

30. The method according to claim 3, wherein the transformation results in the switching off of a lycopene cyclase gene.

31. A genetically modified multinuclear cell of the fungi of the Blakeslea genus, obtained by the method of claim 1.

32. A method for producing carotenoids or their precursors comprising culturing the cells of claim 31 or a mycelium formed therefrom.

33. A method for producing carotenes or xanthophylls comprising culturing the cells of claim 31 or a mycelium formed therefrom.

34. A method for producing astaxanthin, zeaxanthin, echinenone, β-cryptoxanthin, andonixanthin, adonirubin, canthaxanthin, 3-hydroxyechinenone, 3′-hydroxyechinenone, lycopene, β-carotene, α-carotene, lutein, bixin, phytofluene or phytoene comprising culturing the cells of claim 31 or a mycelium formed therefrom.

35. A promoter comprising SEQ ID NO: 1 or SEQ ID NO: 35 for the use in the method according to claim 1.

36. A terminator comprising SEQ ID NO: 2 for the use in the method according to claim 1.

37. A vector comprising SEQ ID NO: 3 for the use in the method according to claim 1.

38. The vector according to claim 37, comprising SEQ ID NO: 69 and/or SEQ ID NO: 70 or SEQ ID NO: 71 and/or SEQ ID NO: 72 or SEQ ID NO: 76.

39. The method according to claim 8, wherein the hygromycin resistance gene (hph) is from E. coli.

40. A genetically modified multinuclear cell of the fungi Blakeslea trispora obtained by the method of claim 1.

41. The method according to claim 1, wherein the method comprises the following additional step after step (i) and before step (ii):
homokaryotic conversion of the cells obtained in step (i) to produce cells in which one or more genetic characteristics of the nuclei are all modified in an identical manner and said genetic modification manifests itself in the cells.
